(19) United States
(12) Patent Application Publication
(10) Pub. No.: US 2023/0122226 A1
(43) Pub. Date: Apr. 20, 2023

(54) CRISPR/CAS9 SYSTEM FOR MULTISTRAIN HIV-1 TREATMENT

(71) Applicant: Board of Regents of the University of Nebraska, Lincoln, NE (US)

(72) Inventors: Howard Gendelman, Omaha, NE (US); Jonathan Herskovitz, Omaha, NE (US); Mahmudul Hasan, Omaha, NE (US)

(21) Appl. No.: 17/905,407

(22) PCT Filed: Mar. 5, 2021
(86) PCT No.: PCT/US2021/021246

§ 371 Date: Aug. 31, 2022

Related U.S. Application Data
(60) Provisional application No. 63/125,545, filed on Dec. 15, 2020.
(60) Provisional application No. 62/986,216, filed on Mar. 6, 2020.
(60) Provisional application No. 62/985,392, filed on Mar. 5, 2020.

(51) Int. Cl. C12N 9/22; C12N 15/86; A61P 31/18

(52) U.S. Cl. CPC C12N 9/22; C12N 15/86; A61P 31/18; C12N 2310/20

(57) ABSTRACT

Nucleic acids for use in CRISPR systems for treating HIV infections are disclosed. Pharmaceutical compositions incorporating the nucleic acids are disclosed as are methods of treating HIV using the nucleic acids.

### CROSS-REFERENCES

This application claims the benefit of priority to U.S. Provisional Application No. 62/985,392, filed on Mar. 5, 2020, U.S. Provisional Application No. 62/986,216, filed on Mar. 6, 2020, and U.S. Provisional Application No. 63/125,545, filed on Dec. 15, 2020, the contents of each are herein incorporated by reference in their entireties.

### STATEMENT OF FEDERAL FUNDING

This invention was made with government support under Grants No. P01 DA028555, P30 MH062261, R01 MH115860, R01 NS034249, R01 NS036126, R01 MH121402, T32 NS105594 all awarded by the National Institutes of Health. The government has certain rights in the invention.

### BACKGROUND

Human immunodeficiency virus infection and acquired immunodeficiency syndrome (HIV/AIDS) is a spectrum of conditions caused by infection with the human immunodeficiency virus (HIV), a retrovirus. Following initial infection a person may not notice any symptoms, or may experience a brief period of influenza-like illness. Typically, this is followed by a prolonged period with no symptoms. If the infection progresses, it interferes more with the immune system, increasing the risk of developing common infections such as tuberculosis, as well as other opportunistic infections, and tumors which are otherwise rare in people who have normal immune function. These late symptoms of infection are referred to as acquired immunodeficiency syndrome (AIDS).

There is no cure or vaccine; however, antiretroviral treatment can slow the course of the disease and may lead to a near-normal life expectancy. Treatment is recommended as soon as the diagnosis is made. Without treatment, the average survival time after infection is 11 years.

In 2019, about 38 million people worldwide were living with HIV and 690,000 deaths had occurred in that year. An estimated 20.6 million of these live in eastern and southern Africa. Between the time that AIDS was identified (in the early 1980s) and 2019, the disease has caused an estimated 32.7 million deaths worldwide.

HIV/AIDS has had a large impact on society, both as an illness and as a source of discrimination. The disease also has large economic impacts

### SUMMARY

Disclosed herein, in certain embodiments, are nucleic acid sequences comprising a crRNA sequence that is complementary to a plurality of nucleic acids of a consensus sequence of an HIV-1 gene selected from the group consisting of: tat, rev, env-gp41, gag-p1, gag-p6, vif, vpr, vpu, and nef. In some embodiments, the nucleic acid sequence comprises two crRNA sequences, each sequence complementary to a plurality of nucleic acids of a consensus sequence of an HIV-1 gene selected from the group consisting of: tat, rev, env-gp41, gag-p1, gag-p6, vif, vpr, vpu, and nef; wherein the crRNA sequences are not complementary to the same sequences. In some embodiments, the crRNA sequence is adjacent to a PAM sequence. In some embodiments, the crRNA sequence is complementary to a plurality of nucleic acids of an overlapping sequence. In some embodiments, the overlapping sequence is part of a nucleic acid sequence of at least two HIV-1 genes selected from the group consisting of: tat, rev, env-gp41, gag-p1, gag-p6, vif, vpr, vpu, and nef. In some embodiments, the overlapping sequence is part of a nucleic acid sequence of at least three HIV-1 genes selected from the group consisting of: tat, rev, env-gp41, gag-p1, gag-p6, vif, vpr, vpu, and nef. In some embodiments, the overlapping exon is part of a nucleic acid sequence selected from the group consisting of tat (exon 1, nucleic acids 5831-6045; exon 2, nucleic acids 8379-8469), rev (exon 1, nucleic acids 5970-6045; or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417). In some embodiments, the overlapping sequence is nucleic acids 7758-8795 of HIV-1 gene gp41-env, exon 2 (nucleic acids 8379-8469) of HIV-1 gene tat, and exon 2 (nucleic acids 8379-8653) of HIV-1 gene rev. In some embodiments, the overlapping exon is exon 1 (nucleic acids 5831-6045) of HIV-1 gene tat, and exon 1 (nucleic acids 5970-6045) of HIV-1 gene rev. In some embodiments, the crRNA has a sequence at least 80%, 85%, 90%, or 95% identical to SEQ ID NO: 1. In some embodiments, the crRNA has a sequence at least 80%, 85%, 90%, or 95% identical to SEQ ID NO: 2. In some embodiments, the crRNA has a sequence at least 80%, 85%, 90%, or 95% identical to SEQ ID NO: 3. In some embodiments, the crRNA has a sequence at least 80%, 85%, 90%, or 95% identical to SEQ ID NO: 4. In some embodiments, the crRNA has a sequence at least 80%, 85%, 90%, or 95% identical to SEQ ID NO: 5. In some embodiments, the crRNA has a sequence at least 80%, 85%, 90%, or 95% identical to SEQ ID NO: 6. In some embodiments, the crRNA has a sequence at least 80%, 85%, 90%, or 95% identical to SEQ ID NO: 7. In some embodiments, the crRNA has a sequence at least 80%, 85%, 90%, or 95% identical to SEQ ID NO: 8. In some embodiments, the crRNA has a sequence according to SEQ ID NO: 1. In some embodiments, the crRNA has a sequence according to SEQ ID NO: 2. In some embodiments, the crRNA has a sequence according to SEQ ID NO: 3. In some embodiments, the crRNA has a sequence according to SEQ ID NO: 4. In some embodiments, the crRNA has a sequence according to SEQ ID NO: 5. In some embodiments, the crRNA has a sequence according to SEQ ID NO: 6. In some embodiments, the crRNA has a sequence according to SEQ ID NO: 7. In some embodiments, the crRNA has a sequence according to SEQ ID NO: 8. In some embodiments, the nucleic acid sequence further comprises a tracrRNA sequence. In some embodiments, the nucleic acid sequence further comprises a sequence that encodes a Cas protein. In some embodiments, the Cas protein is a Cas9, CasPhi (Cas Φ), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas12a (Cpf1), Cas12b, Cas12e, Cas13a, Cas13, Cas13c, or Cas13d. In some embodiments, the Cas protein is a Cas9 protein. In some embodiments, the nucleic acid sequence is a DNA sequence. In some embodiments, the nucleic acid sequence is a RNA sequence. In some embodiments, the nucleic acid sequence further encodes a viral vector. In some embodiments, the viral vector is an adenovirus, an adeno-associated virus (AAV), a retrovirus, or a herpesvirus. In some embodiments, the viral vector is AAV-1, AAV-2, AAV-3, AAV-4, AAV-5, AAV-6, AAV-7, AAV-8, AAV-9, AAV-10, AAV-11, AAV-12, AAV-13 or AAV rh.74. In some embodiments, the viral vector is a lentivirus. In some embodiments, the viral vector is a HIV, FLV, MLV, mMLV, VSV-G enveloped lentivirus, or HIV-enveloped lentivirus. In some embodiments, the viral vector is herpes simplex I virus (HSV). In some embodiments, the nucleic acid further comprises a eukaryotic promoter operably connected to the crRNA sequence. In some embodiments, the nucleic acid comprises tracrRNA sequence and a eukaryotic promoter operably connected to the tracrRNA sequence. In some embodiments, the tracrRNA is operably connected to the crRNA to form a sgRNA. In some embodiments, the nucleic acid comprises a sequence encoding a Cas protein and a eukaryotic promoter operably connected to the Cas protein sequence. In some embodiments, the Cas protein is a Cas9 protein. In some embodiments, the eukaryotic promoter is a cytomegalovirus (CMV) promoter. In some embodiments, the eukaryotic promoter is In some embodiments, the eukaryotic promoter is eukaryotic translation elongation factor 1 alpha (EF-1 alpha).

Disclosed herein, in certain embodiments, are pharmaceutical compositions comprising (a) a nucleic acid disclosed herein, and (b) a pharmaceutically acceptable excipient. In some embodiments, the pharmaceutical composition comprises (a) a nucleic acid comprising TatD and (b) a nucleic acid comprising TatH (TatD/H). In some embodiments, the pharmaceutical composition comprises (a) a nucleic acid comprising TatD and (b) a nucleic acid comprising TatE (TatD/E). In some embodiments, the pharmaceutical composition comprises (a) a nucleic acid comprising TatE and (b) a nucleic acid comprising TatH (TatE/H). In some embodiments, the pharmaceutical composition comprises (a) a nucleic acid comprising TatD and (b) a nucleic acid comprising TatA₂ (TatA₂/D). In some embodiments, the pharmaceutical composition comprises (a) a nucleic acid comprising TatD/tracrRNA and (b) a nucleic acid comprising TatH/tracrRNA. In some embodiments, the pharmaceutical composition comprises (a) a nucleic acid comprising TatD/tracrRNA and (b) a nucleic acid comprising TatE/tracrRNA. In some embodiments, the pharmaceutical composition comprises (a) a nucleic acid comprising TatE/tracrRNA and (b) a nucleic acid comprising TatH/tracrRNA. In some embodiments, the pharmaceutical composition comprises (a) a nucleic acid comprising TatD/tracrRNA and (b) a nucleic acid comprising TatA₂/tracrRNA.

Disclosed herein, in certain embodiments, are methods of disrupting the transcription of an exon of an HIV-1 sequence in an individual in need thereof, comprising administering to the individual a nucleic acid disclosed herein. Disclosed herein, in certain embodiments, are methods of excising all or a portion of an HIV-1 sequence in an individual in need thereof, comprising administering to the individual a nucleic acid disclosed herein. Disclosed herein, in certain embodiments, are methods of treating an HIV-1 infection in an individual in need thereof, comprising administering to the individual a nucleic acid disclosed herein. Disclosed herein, in certain embodiments, are methods of preventing an HIV-1 infection in an individual in need thereof, comprising prophylactically administering to the individual a nucleic acid disclosed herein. Disclosed herein, in certain embodiments, are methods of preventing transmission of an HIV-1 virus from a first individual to a second individual, comprising administering to the first individual a nucleic acid disclosed herein. In some embodiments of a method disclosed herein, the first individual is a pregnant woman and the second individual is a child. In some embodiments of a method disclosed herein, the method comprises administering to the individual (a) a nucleic acid comprising TatD and (b) a nucleic acid comprising TatH (TatD/H). In some embodiments of a method disclosed herein, the method comprises administering to the individual: (a) a nucleic acid comprising TatD and (b) a nucleic acid comprising TatE (TatD/E). In some embodiments of a method disclosed herein, the method comprises administering to the individual: (a) a nucleic acid comprising TatE and (b) a nucleic acid comprising TatH (TatE/H). In some embodiments of a method disclosed herein, the method comprises administering to the individual: (a) a nucleic acid comprising TatD and (b) a nucleic acid comprising TatA₂ (TatA₂/D). In some embodiments of a method disclosed herein, the method comprises administering to the individual (a) a nucleic acid comprising TatD/tracrRNA and (b) a nucleic acid comprising TatH/tracrRNA. In some embodiments of a method disclosed herein, the method comprises administering to the individual (a) a nucleic acid comprising TatD/tracrRNA and (b) a nucleic acid comprising TatE/tracrRNA. In some embodiments of a method disclosed herein, the method comprises administering to the individual (a) a nucleic acid comprising TatE/tracrRNA and (b) a nucleic acid comprising TatH/tracrRNA. In some embodiments of a method disclosed herein, the method comprises administering to the individual (a) a nucleic acid comprising TatD/tracrRNA and (b) a nucleic acid comprising TatA₂/tracrRNA. In some embodiments of a method disclosed herein, a first viral vector comprises the crRNA sequence and the tracrRNA sequence. In some embodiments of a method disclosed herein, the first viral vector comprises a second crRNA sequence, provided that the each crRNA sequence is complementary to a different target sequence. In some embodiments of a method disclosed herein, the crRNA sequence and the tracrRNA sequence are a sgRNA. In some embodiments of a method disclosed herein, a second viral vector comprises the nucleic acid sequences encoding the Cas protein. In some embodiments of a method disclosed herein, the Cas protein is a Cas9, CasPhi (Cas Φ), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas12a (Cpf1), Cas12b, Cas12e, Cas13a, Cas13, Cas13c, or Cas13d. In some embodiments of a method disclosed herein, the Cas protein is a Cas9 protein. In some embodiments of a method disclosed herein, the nucleic acid sequence further encodes a viral vector. In some embodiments of a method disclosed herein, the viral vector is an adenovirus, an adeno-associated virus (AAV), a retrovirus, or a herpesvirus. In some embodiments of a method disclosed herein, the viral vector is AAV-1, AAV-2, AAV-3, AAV-4, AAV-5, AAV-6, AAV-7, AAV-8, AAV-9, AAV-10, AAV-11, AAV-12, AAV-13 or AAV rh.74. In some embodiments of a method disclosed herein, the viral vector is a lentivirus. In some embodiments of a method disclosed herein, the viral vector is a HIV, FLV, MLV, mMLV, VSV-G enveloped lentivirus, or HIV-enveloped lentivirus. In some embodiments of a method disclosed herein, the viral vector is herpes simplex I virus (HSV). In some embodiments of a method disclosed herein, the nucleic acid further comprises a eukaryotic promoter operably connected to the crRNA sequence. In some embodiments of a method disclosed herein, the nucleic acid further comprises a eukaryotic promoter operably connected to the sgRNA sequence. In some embodiments of a method disclosed herein, the nucleic acid further comprises a eukaryotic promoter operably connected to the nucleic acid sequence encoding the Cas protein. In some embodiments of a method disclosed herein, the eukaryotic promoter is a cytomegalovirus (CMV) promoter. In some embodiments of a method disclosed herein, the eukaryotic promoter is a EF-1 alpha.

### BRIEF DESCRIPTION OF DRAWINGS

FIG. 1 exemplifies the exon overlap of HIV-1 genes tat, rev, and env. ARM, arginine rich motif/nuclear localization sequence; OD, oligomerization domain; NES, nuclear export sequence.

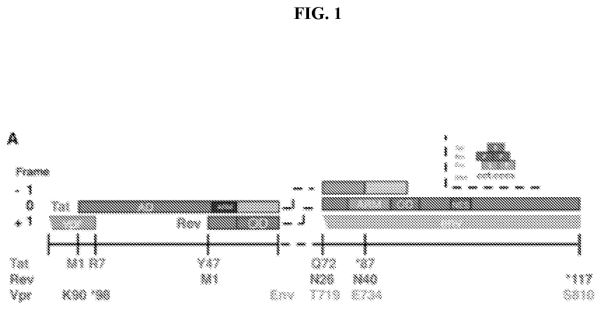

FIG. 2 exemplifies loci of spCas9 mosaic crRNAs. crRNAs directed against HIV-1 LTR/gag or tat exons overlapping with up to two additional genes were designed using an HIV-1 consensus sequence. The crRNA library was individually or dually cloned into an spCas9 expression vector.

FIG. 3 exemplifies CRISPR-Cas9 Targeting HIV-1 tat Consensus Sequence. Sequence logos depicting loci of spCas9 mosaic crRNAs targeting HIV-1 tat exon 1 (nucleotide positions 5831-6045 using HIV-1HXB2 reference strain) and tat exon 2 (nucleotide positions 8379-8469) amongst all curated HIV-1 strain sequences compiled as of 2018 (n=4004; http://www.hiv.lanl.gov/) were generated using WebLogo v3.7.4. Conserved nucleotides are represented as taller letters whereas blank spaces denote frequent gaps in alignment. crRNA directed against sense or antisense strands are shown as rightward (green) or leftward (red) facing arrows, respectively.

FIG. 4 exemplifies mosaic crRNAs reduce HIV-1 replication. A crRNA library was screened against multiple HIV-1 molecular clones via co-transfection of HEK293FT cells for reduction in viral replication as measured by reverse-transcriptase (RT) activity assay. Data depict mean±SEM from four independent experiments each containing biological triplicates.

FIG. 5 exemplifies mosaic crRNA specifically cleave a variety of HIV-1 molecular clones. PCR was performed on DNA extracted from plasmid screening in HEK293FT and PCR amplified single mosaic crRNA or dual crRNA plasmid-treated cells (A) PCR reaction contents were Sanger sequenced and subjected to Inference of CRISPR Edits v2.0 (ICE, Synthego 2020) to (A) quantify editing and (B) visualize nucleotide editing in the PAM/protospacer region.

FIG. 6 exemplifies crRNA induced RT activity reductions correlate with viral sequence conservations. The average percent RT-activity reduction against seven HIV-1 molecular clones was determined. Pearson correlation was determined to be significant when TatE (a positive outlier) is excluded from metanalysis.

FIG. 7 exemplifies TatDE CRISPR Co-transfection Abolishes Latent HIV-1 in Infected ACH2 T cells. (A) ACH2 T cells containing 1 copy of integrated HIV-1 proviral DNA were transfected with 120 ng of CRISPR-Cas9 encoding lentiviral plasmids then stimulated and harvested. (B) Supernatants were measured by RT activity assay 72 hours post stimulation. (C) The fold stimulation in (B) was determined as the ratio of RT activity in the presence:absence of TNFα. (D-E) PCR amplicons from TatD+TatE co-transfection in the presence of TNFα were Sanger sequenced to confirm editing in the TatD and TatE loci. Data in (B-C) depict mean±SEM from three independent experiments each containing biological triplicates. Significance assessed by two- (B) or one-way (C) ANOVA.

FIG. 8 exemplifies TatDE CRISPR Co-transfection Reduces HIV-1 in Latently Infected U1 Promonocytes. (A) U1 promonocytes containing 1-2 copies of integrated HIV-1 proviral DNA were transfected with 120 ng of CRISPR-Cas9 encoding lentiviral plasmids then stimulated and harvested according to the diagram above. (B) Supernatants were measured by RTactivity assay 72 hours post stimulation. (C) The fold stimulation in (B) was determined as the ratio of RT activity in the presence:absence of phorbol 12-myristate 13-acetate (PMA). (D) PCR amplicons from TatD+TatE co-transfection in the presence of PMA were Sanger sequenced to confirm editing in the TatD locus. Data in (B-C) depict mean±SEM from two independent experiments each containing biological triplicates. Significance assessed by two-way ANOVA.

FIG. 9 exemplifies lentiviral TatD+TatE CRISPR co-transduction inactivates latent HIV-1. ACH2 T cells bearing 1 copy of HIV-1 proviral DNA were transduced with lentivirus bearing spCas9-crRNA transgene at multiplicities of infection (MOI) 10, 1, or 0.1. After 72 hours, cells were stimulated with TNFα (15 ng/mL) for 72 additional hours. (A) spCas9 expression as measured by RT-qPCR. (B-D) RT-activity assay from culture supernatants. (F) Sanger sequencing tracings of PCR amplicons. Significance assessed by two-way ANOVA.

FIG. 10 exemplifies that exonic disruption compromises HIV-1 replicative fitness. (A) Insertion/deletion profiles among the most-efficacious single crRNAs from a co-transfection screen were assessed by ICE v2.0 (Synthego) algorithm. Highest frequency insertions or deletions were selected for subsequent non-frameshift site-directed mutagenesis of HIV-1NL4-3-Δnef-eGFP encoding plasmid. (B) HIV-1NL4-3-Δnef-eGFP was mutated in tat/rev loci to match the most frequent indel patterns achieved in single crRNA CRISPR plasmid screening. (C) Transmission electron micrographs of single- or dual-tat mutants. Spherical diameter measurements were taken at the time of imaging (inset). (D-E) CEMss CD4+ T cell lines were challenged at MOI 0.1 with HIV-1NL4-3-Δtat-Δnef-eGFP and assayed at designated time points for RT activity (D) or by flow cytometry for % GFP positive cells (E).

### DETAILED DESCRIPTION

Disclosed herein in certain embodiments are nucleic acid sequences encoding mosaic crRNA sequences. Further disclosed herein, in certain embodiments, are pharmaceutical compositions comprising (a) a nucleic acid sequence encoding a mosaic crRNA sequence, and (b) a pharmaceutically acceptable excipient. Additionally disclosed herein, in certain embodiments, are methods for the treatment and prevention of an HIV infection in an individual in need thereof, comprising administering to the individual a nucleic acid sequence encoding a mosaic crRNA sequence.

### Definitions

Unless defined otherwise, all technical and scientific terms used herein have the same meaning as commonly understood by one of skill in the chemical and biological arts. Additionally, as used in this specification and the appended claims, the singular forms “a”, “an” and “the” include plural referents unless the content clearly dictates otherwise. Thus, for example, reference to “an antibody” includes a plurality of antibodies and reference to “an antibody” in some embodiments includes multiple antibodies, and so forth. Furthermore, unless specifically stated otherwise, the term “about” refers to a range of values plus or minus 10% for percentages (i.e., 10% below that number to 10% above that number) and plus or minus 1.0 unit for unit values, for example, about 1.0 refers to a range of values from 0.9 to 1.1. “About” a range refers to 10% below the lower limit of the range, spanning to 10% above the upper limit of the range.

As used herein, the term “crRNA” means a non-coding short RNA sequence which bind to a complementary target DNA sequence. The crRNA sequence binds to a Cas enzyme (e.g., Cas9) and the crRNA sequence guides the complex via pairing to a specific target DNA sequence.

As used herein, the term “tracrRNA” or trans-activating CRISPR RNA means an RNA sequence that base pairs with the crRNA (to form a functional guide RNA (gRNA)). The tracrRNA sequence binds to a Cas enzyme (e.g., Cas9), while the crRNA sequence of the gRNA directs the complex to a target sequence.

As used herein, the term “gRNA” means the crRNA and a tracrRNA bound together. The gRNA binds to a Cas enzyme (e.g., Cas9) and guides the Cas enzyme to the target sequence.

As used herein, the term “sgRNA” means a single RNA construct comprising a crRNA sequence and a tracrRNA sequence.

As used herein, the term “mosaic crRNAs” mean crRNAs that are constructed from a multiple sequence alignment of separate viral strains, for example separate HIV-1 strains (92UG_029, KER2008, 99KE_KNH1135 etc) or HIV-2 strains.

As used herein, the term “overlapping sequence” or “overlapping exon” means exons or genes that are transcribed in different reading frame from the same part of the DNA sequence.

The terms “individual,” “subject” or “patient,” as used herein, are used interchangeably and mean a mammal, preferably a human, but can also be an animal. None of the terms require the supervision of medical professionals.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with diseases (e.g. cancer). The term “therapeutic effect” refers to the reduction, elimination, or prevention of the disease, symptoms of the disease, or side effects of the disease in the subject.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

crRNAs

Disclosed herein in certain embodiments are mosaic crNRA sequences for the treatment and prevention of HIV infections. In certain embodiments, the crRNA sequences bind to a DNA sequence within an HIV genome (e.g., HIV-1 or HIV-2).

In some embodiments, the crRNAs are “mosaic crRNAs.” In some embodiments, the mosaic crRNA is constructed from a multiple sequence alignment of separate HIV viral strains, for example separate HIV-1 or HIV-2 strains. In some embodiments, the target sequence of the mosaic crRNA is a theoretical composite of an HIV-1 or HIV-2 DNA sequences, for example sequences that retain a high (≥50%) or low (<50%) levels of conservation across isolated HIV strains.

HIV-1 and HIV-2 are two distinct viruses. HIV-1 is the most common HIV virus. HIV-2 occurs in a much smaller number of individual, mostly in individuals found in West Africa. In the U.S., HIV-2 makes up only 0.01% of all HIV cases. The 10 kilobase pair (kb) genome of HIV-1 encodes 3 structural (gag, pol, and env) polyproteins and 6 non-structural (tat, rev, vif, vpu, vpr, and nef) proteins from 3 overlapping alternate reading frames.

HIV-1 has four groups. Group M (Major) accounts for nearly 90% of all HIV-1 cases. HIV-1, group M has nine named strains: A, B, C, D, F, G, H, J, and K. Additionally, Different subtypes can combine genetic material to form a hybrid virus, known as a ‘circulating recombinant form’ (CRFs). HIV-1, group M, strain B strain is the most common strain of HIV in the U.S. Worldwide, the most common HIV strain is HIV-1, group M, strain C. HIV-1 has three additional groups—groups N, O, and P.

In some embodiments, a mosaic crRNA is constructed from a multiple sequence alignment of two or more HIV-1, group M strains selected from: A, B, C, D, F, G, H, J, and K.

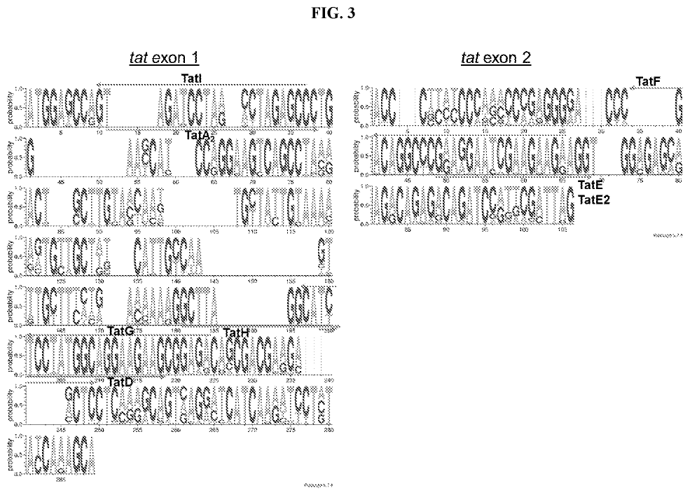

To construct mosaic crRNAs, a consensus HIV sequence can be created. The consensus sequence is based on the most recent alignment for the fullest spectrum of HIV-1 sequences, for example using the Los Alamos National Laboratory database for HIV sequence (hiv.lanl.gov). The Los Alamos database contains 4004 variant sequences. FIG. 3 summarizes the tat locus of all the 4004 sequences; the height of the letters corresponds to percentage of sequences that has that nucleotide in that specific location. For example the first position in FIG. 3 (location 5831 in the HXB2 reference genome) is an A—most of the sequences of the 4004 variants at location 5831 had an A. From all available sequences, a consensus sequence can be generated. Each nucleotide of the consensus sequence can be determined based on being present on most of the sequences, for example is at least 50% of sequences. An exemplary HIV-1 consensus sequence is provided as SEQ ID NO: 9, where N in the consensus sequence means that no nucleotide is present in more 50% of the sequences at that location. SEQ ID NO: 10 provides a consensus sequence for tat exon 1. SEQ ID NO: 11 provides a consensus sequence for tat exon 2.

In some embodiments, a mosaic crRNA disclosed herein binds to a plurality of nucleic acids of an HIV-1 gene selected from the group consisting of: tat, rev, env-gp41, gag-p1, gag-p6, vif, vpr, vpu, and nef. In some embodiments, a mosaic crRNA disclosed herein binds to a plurality of nucleic acids of a gene encoding an HIV-1 protein selected from the group consisting of: Tat, Rev, Env-gp41, Gag-p1, Gag-p6, Vif, Vpr, Vpu, and Nef.

In some embodiments, a mosaic crRNA disclosed herein targets a consensus sequence derived from over 4000 HIV strains in a non-structural multiexon region. In some embodiments, the mosaic crRNA sequence is adjacent to an appropriate PAM sequence. In some embodiments, the mosaic crRNA sequence is adjacent to a S. pyogenes (spCas9) PAM sequence (NGG). In some embodiments, the mosaic crRNA sequence is adjacent to a S. aureus Cas9 (saCas9) PAM sequence (NNGRRT or NGRRN). PAMs for various Cas enzymes are described in Table 1 below, where “N” can be any nucleotide base.

TABLE 1

| CRISPR Nucleases | Organism Isolated From | PAM Sequence (5’ to 3’) |
|---|---|---|
| SpCas9 | Streptococcus pyogenes | NGG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophihis | NNAGAAW |
| LbCpf1 | Lachnospiraceae bacterium | TTTV |
| AsCpf1 | Acidaminococcus sp. | TTTV |

Advantages of the mosaic multiexon cleavage strategy are threefold. (1) First, mosaic crRNAs targeting multiexon regions superiorly reduce viral replication compared to crRNAs targeting LTR promoter DNA and single gene encoding proviral DNA as a result of CRISPR-Cas9 cleavage rather than excision. (2) Second, mosaic crRNAs retain broader coverage against transmitted founder HIV-1 strains compared to conventional CRISPR-Cas9 crRNAs designed against routinely tested laboratory strains of HIV. (3) Third, crRNAs targeting multiexon or regulatory regions display lower likelihood of generating CRISPR-resistant escape mutants.

In some embodiments, a mosaic crRNA disclosed herein binds to a plurality of nucleic acids of an overlapping exon. In some embodiments, the overlapping exon is part of a nucleic acid sequence of at least two HIV-1 genes selected from the group consisting of: tat, rev, env-gp41, gag-p1, gag-p6, vif, vpr, vpu, and nef. In some embodiments, the overlapping exon is part of a nucleic acid sequence of at least three HIV-1 genes selected from the group consisting of: tat, rev, env-gp41, gag-p1, gag-p6, vif, vpr, vpu, and nef. In some embodiments, In some embodiments, the overlapping exon is part of a nucleic acid sequence of HIV-1 genes tat, rev, and env.

In some embodiments, a mosaic crRNA disclosed herein binds to a plurality of nucleic acids of a HIV-1 sequence (HXB2, complete genome; HIV1/HTLV-III/LAV reference genome; GenBank: K03455.1) selected from: tat (exon 1, nucleic acids 5831-6045; exon 2, nucleic acids 8379-8469), rev (exon 1, nucleic acids 5970-6045; or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417).

In some embodiments, the mosaic crRNA is selected from a crRNA of Table 2 below:

TABLE 2

| crRNA | Target DNA Complementary | crRNA Seed Sequence |
|---|---|---|
| Name | Sequence (5’→3’) | (5’→3’) |
| TatA₂ | TAGATCCTAACCTAGAGCCC | UAGAUCCUAACCUAGAGCCC (SEQ ID |
| | | NO. 1) |
| TatD | TCTCCTATGGCAGGAAGAAG | UCUCCUAUGGCAGGAAGAAG (SEQ ID |
| | | NO: 2) |
| TatE | GAAGGAATCGAAGAAGAAGG | GAAGGAAUCGAAGAAGAAGG (SEQ ID |
| | | NO: 3) |
| TatE₂ | GAAAGAATCGAAGAAGGAGG | GAAAGAAUCGAAGAAGGAGG (SEQ ID |
| | | NO: 4) |
| TatF | CCGATTCCTTCGGGCCTGTC | CCGAUUCCUUCGGGCCUGUC (SEQ ID |
| | | NO: 5) |
| TatG | TCTCCGCTTCTTCCTGCCAT | UCUCCGCUUCUUCCUGCCAU (SEQ ID |
| | | NO: 6) |
| TatH | GCTTAGGCATCTCCTATGGC | GCUUAGGCAUCUCCUAUGGC (SEQ ID |
| | | NO: 7) |
| TatI | GGCTCTAGGTTAGGATCTAC | GGCUCUAGGUUAGGAUCUAC (SEQ ID |
| | | NO: 8) |

In some embodiments, the mosaic crRNA is TatA₂—UAGAUCCUAACCUAGAGCCC (SEQ ID NO. 1). In some embodiments, the mosaic crRNA is TatD—UCUCCUAUGGCAGGAAGAAG (SEQ ID NO: 2). In some embodiments, the mosaic crRNA is TatE—GAAGGAAUCGAAGAAGAAGG (SEQ ID NO: 3). In some embodiments, the mosaic crRNA is TatE₂—GAAAGAAUCGAAGAAGGAGG (SEQ ID NO: 4). In some embodiments, the mosaic crRNA is TatF—CCGAUUCCUUCGGGCCUGUC (SEQ ID NO: 5). In some embodiments, the mosaic crRNA is TatG—UCUCCGCUUCUUCCUGCCAU (SEQ ID NO: 6). In some embodiments, the mosaic crRNA is TatH—GCUUAGGCAUCUCCUAUGGC (SEQ ID NO: 7). In some embodiments, the mosaic crRNA is TatI—GGCUCUAGGUUAGGAUCUAC (SEQ ID NO: 8).

In some embodiments, the mosaic crRNA is 80%, 85%, 90%, or 95% identical to TatA₂—UAGAUCCUAACCUAGAGCCC (SEQ ID NO. 1). In some embodiments, the mosaic crRNA is 80%, 85%, 90%, or 95% identical to TatD—UCUCCUAUGGCAGGAAGAAG (SEQ ID NO: 2). In some embodiments, the mosaic crRNA is 80%, 85%, 90%, or 95% identical to TatE—GAAGGAAUCGAAGAAGAAGG (SEQ ID NO: 3). In some embodiments, the mosaic crRNA is 80%, 85%, 90%, or 95% identical to TatE₂—GAAAGAAUCGAAGAAGGAGG (SEQ ID NO: 4). In some embodiments, the mosaic crRNA is 80%, 85%, 90%, or 95% identical to TatF—CCGAUUCCUUCGGGCCUGUC (SEQ ID NO: 5). In some embodiments, the mosaic crRNA is 80%, 85%, 90%, or 95% identical to TatG—UCUCCGCUUCUUCCUGCCAU (SEQ ID NO: 6). In some embodiments, the mosaic crRNA is 80%, 85%, 90%, or 95% identical to TatH—GCUUAGGCAUCUCCUAUGGC (SEQ ID NO: 7). In some embodiments, the mosaic crRNA is 80%, 85%, 90%, or 95% identical to TatI—GGCUCUAGGUUAGGAUCUAC (SEQ ID NO: 8).

In some embodiments, a mosaic crRNA disclosed herein reduces HIV-1 replication by at least 50%. In some embodiments, a mosaic crRNA disclosed herein reduces HIV-1 replication by at least 60%, 65%, 70%, 75%, 80%, 85%, 90%, 95%, or 99%. In some embodiments, TatD reduces HIV-1 replication by at least 54%. In some embodiments, TatE reduces HIV-1 replication by 76%. In some embodiments, co-administration of TatD and TatE (TatDE) reduces HIV-1 replication by an average of 82% in 7 strains, including 6 clade B transmitted founder strains.

In some embodiments, a mosaic crRNA disclosed herein is effective against at least 50% of HIV-1 strains. In some embodiments, a mosaic crRNA disclosed herein is effective against at least 60%, 65%, 70%, 75%, 80%, 85%, 90%, 95%, or 99% of HIV-1 strains. In some embodiments, TatDE therapy is effective against at least 62% of all HIV-1 strains.

In some embodiments, a crRNA disclosed herein is operable with any suitable Cas enzyme. In some embodiments, a crRNA disclosed herein is operable with a Cas enzyme selected from the group consisting of: Cas9, CasPhi (Cas Φ), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas12a (Cpf1), Cas12b, Cas12e, Cas13a, Cas13, Cas13c, and Cas13d. In some embodiments, a crRNA disclosed herein is operable with Cas9.

In some embodiments, a crRNA disclosed herein is part of a single guide RNA (“sgRNA”) sequence wherein the sgRNA sequence comprises the crRNA sequences and a tracrRNA sequence. Any suitable tracrRNA sequence is contemplated for use with a sgRNA disclosed herein. In some embodiments, the sgRNA comprises TatA₂ and a tracrRNA sequence. In some embodiments, the sgRNA comprises TatD and a tracrRNA sequence. In some embodiments, the sgRNA comprises TatE and a tracrRNA sequence. In some embodiments, the sgRNA comprises TatE₂ and a tracrRNA sequence. In some embodiments, the sgRNA comprises TatF and a tracrRNA sequence. In some embodiments, the sgRNA comprises TatG and a tracrRNA sequence. In some embodiments, the sgRNA comprises TatH and a tracrRNA sequence. In some embodiments, the sgRNA comprises TatI and a tracrRNA sequence.

In some embodiments, the crRNA sequence is a DNA sequence (such as single- or double stranded linear sequences; or plasmid DNA), an RNA sequence, or a recombinantly expressed crRNA/protein fusion (such as ribonucleoprotein (RNP)). In some embodiments, the DNA or RNA sequence comprising the crRNA sequence further comprises a tracrRNA sequence (e.g., a sgRNA sequence) and/or a sequence encoding a Cas9 enzyme.

### Delivery Vehicles

In some embodiments, a crRNA or sgRNA disclosed herein is part of any suitable delivery vehicle. In some embodiments, the delivery vehicle is a plasmid. In some embodiments, the delivery vehicle is a viral vector (e.g., a recombinant viral vector). Any suitable plasmid or viral vector is contemplated for use with the crRNA and/or sgRNA sequences disclosed herein.

In some embodiments, the delivery vehicle is a viral vector. In some embodiments, a single viral vector comprises a crRNA sequence disclosed herein, a tracrRNA sequence, and a sequence encoding a Cas enzyme. In some embodiments, the crRNA sequence, tracrRNA sequence, and sequence encoding the Cas enzyme are spilt across multiple vectors (e.g., 2 or 3 vectors).

In some embodiments, the viral vector is genetically modified from its wildtype counterpart (i.e., a recombinant viral vector). For example, the viral vector comprises an insertion, deletion, or substitution of one or more nucleotides, for example to facilitate cloning or change the properties of the viral vector. Such properties may include packaging capacity, transduction efficiency, immunogenicity, genome integration, replication, transcription, and translation. In some embodiments, a portion of the viral genome is deleted such that the virus is capable of packaging exogenous sequences having a larger size. In some embodiments, the viral vector has enhanced transduction efficiency. In some embodiments, the immune response induced by the virus in a host is reduced. In some embodiments, viral genes (such as, e.g., integrase) that promote integration of the viral sequence into a host genome are mutated such that the virus is non-integrating. In some embodiments, the viral vector is replication defective. In some embodiments, the viral vector comprises exogenous transcriptional or translational control sequences to drive expression of coding sequences on the vector. In some embodiments, a viral vector is modified to target a particular tissue or cell type. For example, viral surface proteins may be altered to decrease or eliminate viral protein binding to its natural cell surface receptor(s). The surface proteins may also be engineered to interact with a receptor specific to a desired cell type.

In some embodiments, the recombinant viral vector is an adenovirus (such as Ad26 and Ad5), adeno-associated viral vectors (such as scAAV, AAV-DJ/8, AAV1, AAV2, AAV4, AAV5, AAV6, AAV8, and AAV9), a helper-dependent adenovirus, a retroviral vector (such as a lentivirus; more particularly, HIV, FLV, MLV, mMLV, VSV-G enveloped lentivirus, HIV-enveloped lentivirus), a hemagglutinating virus of Japan-liposome (HVJ), an orthopox or avipox vector, a herpesvirus vectors (e.g., a herpes simplex I virus (HSV) vector), a bacteriophage T4, or a baculovirus.

In some embodiments, the viral vector is an AAV vector. In some embodiments, the viral vector may a lentivirus vector. In some embodiments, the lentivirus may be non-integrating. In some embodiments, the viral vector may be an adenovirus vector. In some embodiments, the adenovirus is a high-cloning capacity or “gutless” adenovirus, where all coding viral regions apart from the 5′ and 3′ inverted terminal repeats (ITRs) and the packaging signal (Y) are deleted from the virus to increase its packaging capacity. In some embodiments, the viral vector is an HSV-1 vector. In some embodiments, the HSV-1-based vector is helper dependent, and in other embodiments it is helper independent. In some embodiments, the viral vector is a bacteriophage T4. In some embodiments, the bacteriophage T4 may be able to package any linear or circular DNA or RNA molecules when the head of the virus is emptied. In some embodiments, the viral vector is a baculovirus vector. In some embodiments, the viral vector is a retrovirus vector.

In embodiments using AAV or lentiviral vectors, which have smaller cloning capacity, it may be necessary to use more than one vector to deliver all the components of a vector system as disclosed herein. For example, one AAV vector may contain sequences encoding a Cas9 protein, while a second AAV vector may contain one or more guide sequences and one or more copies of donor polynucleotide.

In some embodiments, the viral vector is a recombinant adeno-associated virus (AAV) vector. Any suitable AAV is contemplated for use with the crRNAs and sgRNAs disclosed herein. In some embodiments, the AAV is AAV-1, AAV-2, AAV-3, AAV-4, AAV-5, AAV-6, AAV-7, AAV-8, AAV-9, AAV-10, AAV-11, AAV-12, AAV-13 and AAV rh.74.

In some embodiments, the recombinant viral vector comprises a eukaryotic promoter operably linked to the crRNA or sgRNA. Any suitable promoter is contemplated for use with the vectors disclosed herein. In some embodiments, the promoter is a cytomegalovirus (CMV) promoter. In some embodiments of a method disclosed herein, the eukaryotic promoter is a EF-1 alpha. Other suitable promoters which may be used include, but are not limited to, the Rous sarcoma virus (RSV), the SV40 early promoter region, the herpes thymidine kinase promoter, the regulatory sequences of the metallothionein (MMT) gene, prokaryotic expression vectors such as the β-lactamase promoter, the tac promoter, promoter elements from yeast or other fungi such as the Gal 4 promoter, the ADC (alcohol dehydrogenase) promoter, PGK (phosphoglycerol kinase) promoter, alkaline phosphatase promoter;

In some embodiments, the recombinant viral vector is replication-defective. A replication-defective viral vector requires the deleted function(s) to be provided in trans during viral replication and encapsidation (by using, e.g., a helper virus or a packaging cell line carrying gene products necessary for replication and/or encapsidation).

In some embodiments, the delivery vehicle is a nanoformulations (e.g. lipid nanoparticles (LNPs), mesoporous nanoparticles (MNPs)), viral receptor targeted nanoformulation, pseudovirion of HIV-1 and gammaretroviral vectors. In some embodiments, a delivery vehicle disclosed herein comprises a targeting moiety such as a targeting moiety that targets cell surface receptors present on HIV-1 infected cells.

In other embodiments, multiple vectors are used to administer the crRNA/tracr/Cas sequences (i.e., a vector system). In some embodiments, the vector system comprises two vectors. In some embodiments, the vector system comprises three vectors. In some embodiments, the vector system comprises a vector comprising a crRNA disclosed herein (any of TatA₂, TatD, TatE, TatE₂, TatF, TatG, TatH, or TatI) and a tracrRNA, or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA). If the vector comprising the crNRA sequence does not comprise tracrRNA, then in some embodiments a vector separately comprises a tracrRNA. In some embodiments, the vector system comprises a vector comprising a nucleotide sequence encoding a Cas protein (e.g., a Cas9 protein).

When encoded by one or more viral vectors, the crRNA, tracr and Cas sequences may be oriented in the same or different directions and in any order on the vector.

A crRNA disclosed herein (any of TatA₂, TatD, TatE, TatE₂, TatF, TatG, TatH, or TatI) or sgRNA (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) disclosed herein can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes.

In some embodiments, a crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) is formulated as a lipid nanoparticle (LNP). A LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm.

LNPs may be made from cationic, anionic, or neutral lipids. Neutral lipids, such as the fusogenic phospholipid DOPE or the membrane component cholesterol, may be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs may also comprise hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

### Compositions

Disclosed herein, in certain embodiments, are pharmaceutical compositions comprising (a) a crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA), and (b) a pharmaceutically-acceptable excipient. In some embodiments, the composition further comprises a Cas enzyme.

In some embodiments, the composition comprises: TatD and TatH (TatD/H). In some embodiments, the composition comprises: TatD and TatE (TatD/E). In some embodiments, the composition comprises: TatE and TatH (TatE/H). In some embodiments, the composition comprises: TatD and TatA₂ (TatA₂/D).

In some embodiments, the composition comprises: TatD/tracrRNA and TatH/tracrRNA. In some embodiments, the composition comprises: TatD/tracrRNA and TatE/tracrRNA. In some embodiments, the composition comprises: TatE/tracrRNA and TatH/tracrRNA. In some embodiments, the composition comprises: TatD/tracrRNA and TatA₂/tracrRNA.

In some embodiments, a crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) is part of a viral vector. In some embodiments, the Cas enzyme is part of a viral vector. In some embodiments, the crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) and the Cas enzyme are part of the same viral vector. In some embodiments, the crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) and the Cas enzyme are part of separate viral vectors.

In some embodiments, the pharmaceutically-acceptable excipient is a carrier, solvent, stabilizer, adjuvant, diluent, etc., depending upon the particular mode of administration and dosage form.

In some embodiments, the composition has a physiologically compatible pH (e.g., a range from a pH of about 3 to a pH of about 11, about pH 3 to about pH 7, depending on the formulation and route of administration). In some cases, the pH is from about pH 5.0 to about pH 8.

In some embodiments, the composition further comprises a second active ingredient useful in the treatment or prevention of bacterial growth (for example and without limitation, anti-bacterial or anti-microbial agents).

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, cellulose, dextrin).

### Methods of Use

Disclosed herein, in certain embodiments, are methods of treating an HIV-1 infection in an individual in need thereof. Further disclosed herein, in certain embodiments, are methods of preventing an HIV-1 infection in an individual in need thereof. Additionally, disclosed herein, in certain embodiments, are methods of preventing transmission of an HIV-1 virus from one individual to another (for example, from a pregnant woman to a child, for example during birth or breast feeding).

In some embodiments, the methods comprise administering to an individual a crRNA disclosed here (any of TatA₂, TatD, TatE, TatE₂, TatF, TatG, TatH, or TatI). In some embodiments, the methods comprise administering to an individual any combination of a crRNA disclosed here. In some embodiments, the method comprises administering to the individual: TatD and TatH (TatD/H). In some embodiments, the method comprises administering to the individual: TatD and TatE (TatD/E). In some embodiments, the method comprises administering to the individual: TatE and TatH (TatE/H). In some embodiments, the method comprises administering to the individual: TatD and TatA₂ (TatA₂/D).

In some embodiments, the methods comprise administering to an individual any sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA). In some embodiments, the methods comprise administering to an individual any combination of a sgRNA disclosed here. In some embodiments, the method comprises administering to the individual: TatD/tracrRNA and TatH/tracrRNA. In some embodiments, the method comprises administering to the individual: TatD/tracrRNA and TatE/tracrRNA. In some embodiments, the method comprises administering to the individual: TatE/tracrRNA and TatH/tracrRNA. In some embodiments, the method comprises administering to the individual: TatD/tracrRNA and TatA₂/tracrRNA.

In some embodiments, the method dysregulates virion production from a latent proviral DNA or impede integration of reverse-transcribed proviral DNA. In some embodiments, the crRNA is a mosaic crRNA. In some embodiments, the crRNA binds to a plurality of nucleic acids of an overlapping exon of at least two HIV-1 genes. In some embodiments, the crRNA binds to a plurality of nucleic acids of an overlapping exon of at least three HIV-1 genes.

Further disclosed herein, in certain embodiments, are methods of excising all or a portion of an HIV genome in an HIV infected cell of an individual. In some embodiments, the method comprises administering to the individual a first a crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) that binds to a first HIV sequence and a second a crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) that binds to a second HIV sequence, provided that the first crRNA or sgRNA and the second crRNA or sgRNA are different crRNAs or sgRNAs. In some embodiments, at least one of the first crRNA and the second crRNA is a mosaic crRNA. In some embodiments, at least one of the first crRNA and the second crRNA binds to a plurality of nucleic acids of an overlapping exon of at least two HIV-1 genes. In some embodiments, at least one of the first crRNA and the second crRNA binds to a plurality of nucleic acids of an overlapping exon of at least three HIV-1 genes.

A pharmaceutical composition disclosed herein is administered by any appropriate route that results in effective treatment in the subject. In some embodiments, a pharmaceutical composition disclosed herein is administered systemically. In some embodiments, a pharmaceutical composition disclosed herein is administered locally. The pharmaceutical composition is administered via a route such as, but not limited to, enteral, gastroenteral, oral, transdermal, subcutaneous, nasal, intravenous, intravenous bolus, intravenous drip, intraarterial, intramuscular, transmucosal, insufflation, sublingual, buccal, conjunctival, cutaneous. Modes of administration include injection, infusion, instillation, and/or ingestion. “Injection” includes, without limitation, intravenous, intramuscular, intraarterial, intrathecal, intraventricular, intradermal, intraperitoneal, transtracheal, and subcutaneous. In some examples, the route is intravenous.

### EXAMPLES

### Example 1

We have devised a CRISPR-Cas9 approach that utilizes mosaic crRNA to achieve exonic disruption, rather than pure excision, to suppress HIV-1 replication. Using this technology, integration of HIV complementary DNA (cDNA) and proviral DNA transcription can be blocked. The 10 kilobase pair (kb) genome of HIV-1 encodes 3 structural (gag, pol, and env) polyproteins and 6 non-structural (tat, rev, vif, vpu, vpr, and nef) proteins from 3 overlapping alternate reading frames. As such, mosaic crRNAs have been devised that target consensus sequences derived from over 4000 HIV strains in these non-structural multiexon regions. Advantages of the mosaic multiexon cleavage strategy are threefold. (1) First, mosaic crRNAs targeting multiexon regions superiorly reduce viral replication compared to crRNAs targeting LTR promoter DNA and single gene encoding proviral DNA as a result of CRISPR-Cas9 cleavage rather than excision. (2) Second, mosaic crRNAs retain broader coverage against transmitted founder HIV-1 strains compared to conventional CRISPR-Cas9 crRNAs designed against routinely tested laboratory strains of HIV. (3) Third, crRNAs targeting multiexon or regulatory regions display lower likelihood of generating CRISPR-resistant escape mutants.

A summary of our findings is provided herein:

1. Our top mosaic crRNA candidates, TatD and TatE, individually reduce HIV-1 replication by 54% and 76%, respectively.

2. In the absence of antiretroviral therapy, tandem TatD and TatE (TatDE) CRISPR therapy reduces HIV-1 replication by an average of 82% in 7 strains, including 6 clade B (prevailing subtype in Europe, Australia, and North America) transmitted founder strains.

3. TatDE therapy is predicted to work against at least 62% of all HIV-1 strains; this assumes no mismatch tolerance between mosaic crRNAs and protospacer targets.

4. Lentiviral transduction of TatDE therapy in latently HIV-1 infected ACH2 T cells blocks latency reversal by 94%.

5. No off-target editing was observed in algorithm selected putative off-target loci in the human genome.

6. Point mutagenesis at the TatE locus paralleling CRISPR edits, halted viral replicative fitness for 28 days; this suggests lack of escape mutants after CRISPR therapy.

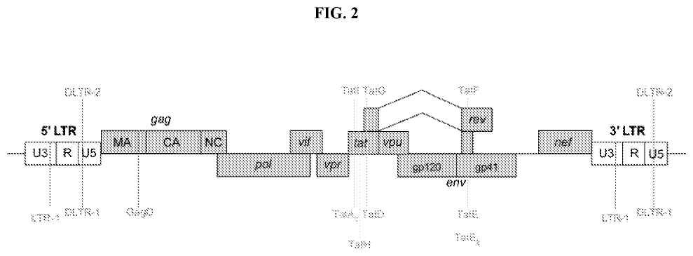

To achieve these objectives, we analyzed the HIV-1 genome for accessory protein genes that play critical roles in regulating the viral lifecycle. HIV transactivator of transcription (Tat) binds to the host transcription machinery and promote elongation of the full-length transcripts by RNA Polymerase II. HIV Rev is an accessory protein that helps migration of viral complete or spliced mRNA transcripts from the nucleus to the cytoplasm of cells. In the absence of Rev, mRNA of structural genes like gag and gag-p1 are retained in the nucleus and are not translated. Because Tat and Rev are encoded from the same DNA sequence but in different reading frames, we selected crRNAs that could simultaneously cleave DNA sequences encoding both proteins. HIV env gene also partially overlaps with tat and rev DNA segments. Thus, some of our Tat-directed crRNAs may adversely impact the functionality of HIV envelope (FIG. 2).

In addition, we designed mosaic crRNAs against tat regions that are conserved in 6-67% of 4004 known HIV-1 strains (Table 3), compiled as a consensus sequence that weights a multiple sequence alignment obtained from the Los Alamos National Laboratory database (FIG. 3). Previous dual crRNA LTR-gag excision and Drexel University LTR-targeted CRISPR-Cas9 systems to offer similar coverage against the broad diversity of HIV-1 sequences (Table 3). Furthermore, our crRNAs were predicted by CasOFFinder tool to exhibit similar theoretical off-target toxicity in human genomes than the aforementioned HIV-1 targeting CRISPR-Cas9 systems (Table 4).

TABLE 3

| | | % Sequence | |
|---|---|---|---|
| | Target DNA | Conservation | CRISPRspec |
| crRNA Name | Sequence (5’→3’) | (n = 4004) | Score‡ |
| LTR-1 (control) | GCAGAACTACACACCAGGGCC | 27.12% | 6.937 |
| GagD (control) | GATAGATGTAAAAGACACCA | 11.14% | 4.071 |
| DLTR-1 (control) | GACTGCTTAAGCCTCAATAA | 29.57% | 3.364 |
| DLTR-2 (control) | GCTTTATTGAGGCTTAAGCAG | 28.94% | 2.266 |
| TatG | TCTCCGCTTCTTCCTGCCAT | 67.15% | 3.803 |
| TatD | TCTCCTATGGCAGGAAGAAG | 59.04% | 6.284 |
| TatH | GCTTAGGCATCTCCTATGGC | 51.12% | 4.662 |
| TatA₂ | TAGATCCTAACCTAGAGCCC | 29.79% | 7.327 |
| TatI | GGCTCTAGGTTAGGATCTAC | 25.90% | 6.844 |
| TatE | GAAGGAATCGAAGAAGAAGG | 15.26% | 3.090 |
| TatF | CCGATTCCTTCGGGCCTGTC | 6.19% | 6.467 |
| TatE₂ | GAAAGAATCGAAGAAGGAGG | 5.82% | 6.021 |

‡CRISPRspec score represents the specificity of the selected crRNA for HIV-1 based on a predefined off-target landscape within the human genome. Calculated using CRISPRoff webserver v 1.1.

Conserved regions of the HIV-1 genome include portions of flanking LTRs, gp120-encoding segments of env, and multiple exon overlaps (FIG. 2). Low entropy in HIV-1 genes encoding p6/protease, tat/rev, vpu/gp120, and nef/3′ LTR highlight sequence preservation in loci that govern multiple phases of the viral lifecycle.

A library of eight HIV-1 tat-targeting crRNAs designed for deployment with Streptococcus pyogenes Cas9 (spCas9) endonuclease were created. These “mosaic” crRNAs were constructed against the tat consensus sequence synthesized from 4004 HIV-1 strains. Five of the eight produced crRNAs target the sense strand. Because portions of tat overlap with rev and the gp41 portion of env, up to three exons could be simultaneously disrupted with these crRNAs. Notably, tat-directed crRNAs retain full 20 bp complementarity for 6-67% of all known HIV-1 strains. Duplexed mosaic crRNA treatments were found to target at least 56-62% of strains, assuming no nucleotide mismatch tolerance. Taken together, these data demonstrate that crRNAs can be produced with broad HIV-1 excision potential while, at the same time, limiting off-target effects.

Our tat-targeted mosaic crRNA library was cloned onto spCas9 expression vector pX333 (Addgene #64073) and screened for breadth of HIV-1 strain cleavage.

As exhibited in FIG. 3, TatD and TatE each reduce viral replication against the majority of HIV-1 strains tested (n=7), which include 6 clinically derived transmitted/founder strain molecular clones (NIH ARP #11919). TatD simultaneously targets the first exons of tat (exon 1, nucleic acids 5831-6045) and rev (exon 1, nucleic acids 5970-6045), whereas TatE targets gp41-env (nucleic acids 5970-6045; or exon 2, nucleic acids 8379-8653), env-gp41 ( ) and the second exons of tat (exon 2, nucleic acids 8379-8469) and rev (exon 2, nucleic acids 8379-8653) (FIG. 2).

To test the hypothesis that conserved crRNAs would most effectively limit viral replication, a plasmid co-transfection screen was implemented. Human embryonic kidney (HEK293FT) cells were transfected with plasmids encoding HIV-1 in the absence (untreated control) or presence of spCas9-crRNA constructs. Seven HIV-1 molecular clones, one laboratory (NL4-3) plus six clade B founder strains, were included to reflect predominant North American and European viral subtype sequence heterogeneity. Four independent co-transfection screens were performed to ensure experimental accuracy. Supernatants were measured after 72 hours for HIV reverse transcriptase (RT) activity.

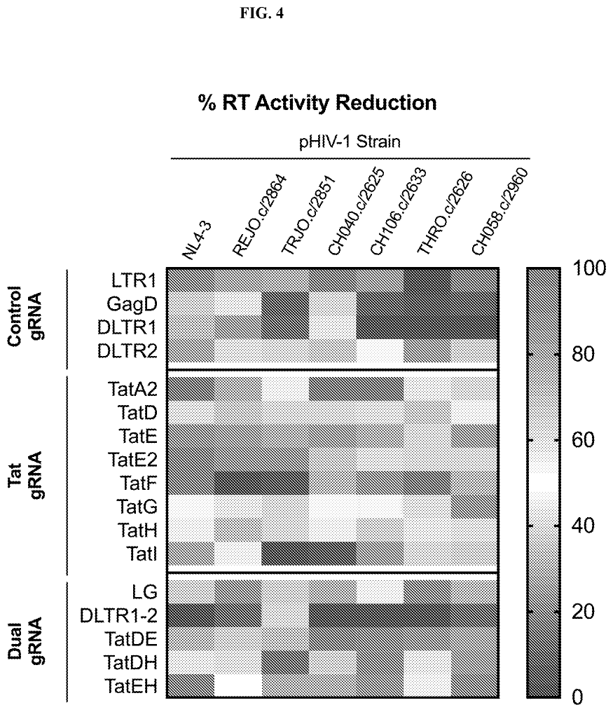

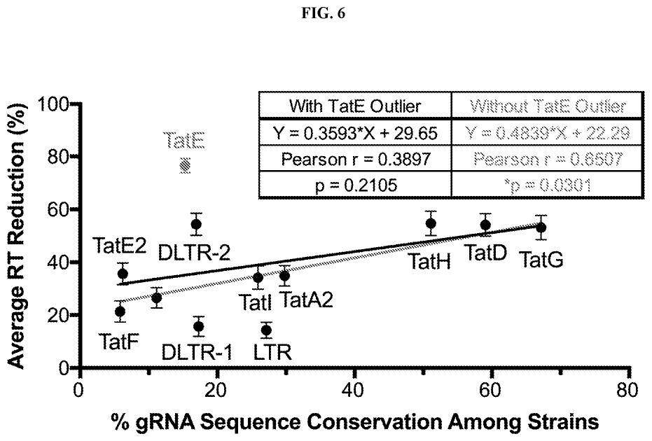

The single mosaic crRNA CRISPR TatE and TatD constructs demonstrated the greatest suppression of HIV-1 replication (FIG. 4). TatE crRNA reduced RT activity by an average of 76% (±6.5%, standard error of the mean (SEM)), with more than 75% reduction in 6 of 7 tested HIV-1 strains. Notably, TatE outperformed all single crRNA controls, including DLTR-2. Pearson correlation analysis evaluated whether CRISPR crRNA activity was associated with target sequence conservation (FIG. 6). While positive trends were observed, significance was achieved when TatE was excluded from analysis. The data support the idea that although crRNA targeting conserved genomic regions of HIV-1 proviral DNA are generally more efficacious, the TatE locus is critical to viral replication.

We next sought to determine whether proviral DNA excision induced by two CRISPR-Cas9 crRNAs would further suppress HIV-1 replication. The noted efficacy of TatE, which targets a region of tat that overlaps with both rev and env, prompted us to posit that simultaneous disruptions of multiple viral exons would most drastically blunt HIV-1's lifecycle. Plasmids were subcloned to express various combinations of the top three performing tat-directed crRNAs. Table 5 summarizes the percent conservation, excision fragment length, and number of deactivated exons elicited by each pairing. Increased antiretroviral activity was observed when TatD and TatE crRNAs were co-expressed (TatDE). Viral replication in TatDE-treated cells were lowered on average by 82% (±4.6% SEM), with a range of 65-98% across all tested viral strains (FIG. 4). TatDE demonstrated greater levels of viral suppression compared to LTR-1/GagD (“LG”) and DLTR-1/DLTR-2 (“DLTR1-2”) duplexed reference CRISPR controls by measured RT activity. The ˜2.5 kilobase pair (kb) dropout of intervening proviral DNA was observed against all seven HIV-1 molecular clones. To confirm the accuracy of TatDE CRISPR, excision bands were Sanger sequenced and analyzed by Inference of CRISPR Edits (ICE) algorithm. Sequencing data qualitatively depict nucleotide degeneracy in both target loci, with insertion/deletion (indel) mutations at a rate of 25-51% in excision bands. Pairing our two most conserved efficacious crRNAs, TatDH, and of our largest excising duplex, TatEH, proved inferior to 5-exon inactivating TatDE in suppressing RT activity. Taken together, these data support the hypothesis that disrupting a maximal number of viral exons by CRISPR-Cas9 leads to the greatest suppression of HIV-1 replication.

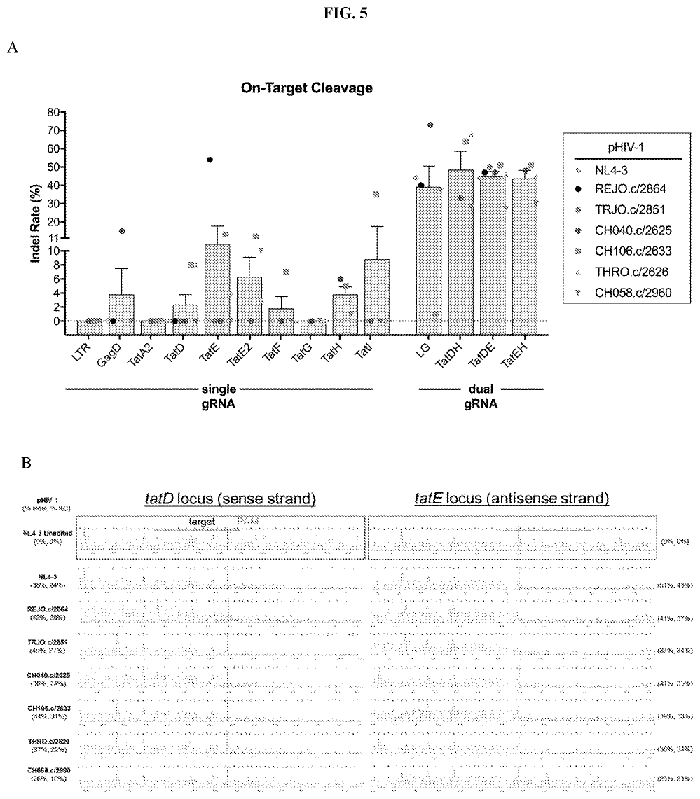

We next evaluated the specificity of our tat-directed CRISPR-Cas9 by comparing the degree of proviral genome editing to that observed in off-target loci. ICE analysis performed on Sanger sequences from these reactions revealed that dual crRNA systems improve indel mutation rates to >40% as compared to <10% seen with single crRNA controls (FIG. 5). TatDE therapy resulted in an average of 45% (±3.0% SEM) gene modification rate with 40-50% editing in 6 of 7 tested strains, supporting TatDE's ability to deactivate a broad variety of HIV-1 proviral species. Multiplexing TatD and TatE, improved antiviral efficacy to 82% on average (FIG. 4). A positive association was identified between crRNA target conservation and antiviral efficacy when TatE, a positive outlier, was excluded (FIG. 6).

To ensure TatDE cleavage is restricted to HIV-1 proviral DNA, we investigated the potential for aberrant indels at off-target loci in the human genome. These covered 5 possible loci recognized by each TatD, TatE, LTR-1, and GagD. At all putative off-target positions for TatD and TatE, no editing was observed (Table 4). This was sustained regardless of whether cells were treated with single or dual crRNAs. Similarly, off-target analysis by ICE for LTR-1 and GagD failed to demonstrate indel mutations in host genes. Seven of 10 possible off-target regions for TatDE fell in intronic regions whereas only 2 of 10 for LTR-1/GagD were noncoding. These results reinforce the notion that TatDE CRISPR is not more likely to affect host gene expression than other HIV-1 CRISPR systems.

TABLE 4
TatDE Off-Target Cleavage

| | | | | % Indel (per pHIV-1 Strain) | | | | |
|---|---|---|---|---|---|---|---|---|
| | Guide Target | | | pCHO | pCH1 | pTH | pCHO | |
| | (Protospacer | Gene | Treat- | 40.c/ | 06.c/ | RO.c/ | 58.c/ | Gene |
| crRNA | mismatches) + PAM | (Locus) | ment | 2625 | 2633 | 2626 | 2960 | Function |
| Tat | CCTCCCATGGCAGA | ALDH3B1 | TatD | 0% | 0% | 0% | 0% | Aldehyde |
| D | AAGAAGTGG | (11q13.2) | TatDE | 0% | 0% | 0% | 0% | dehydrogenase |
| | | | | | | | | aids in alcohol |
| | | | | | | | | metabolism |
| | | | | | | | | and lipid |
| | | | | | | | | peroxidation |
| | | | | | | | | (Accession |
| | | | | | | | | NG_012282) |
| | CTTCCCACGGCAGG | GPC6 | TatD | 0% | 0% | 0% | 0% | Glypicans |
| | AAGAACCGG | (13q31.3- | TatDE | 0% | 0% | 0% | 0% | encode cell |
| | | q32.1) | | | | | | surface |
| | | | | | | | | coreceptors for |
| | | | | | | | | control of cell |
| | | | | | | | | growth and |
| | | | | | | | | division |
| | | | | | | | | (Accession |
| | | | | | | | | NG_011880) |
| | CTTCCTAGGGCAGG | IGFBP5 | TatD | 0% | 0% | 0% | 0% | Insulin-like |
| | AAGAAGGGG | (2q35) | TatDE | 0% | 0% | 0% | 0% | growth factor- |
| | | | | | | | | binding |
| | | | | | | | | protein 5; |
| | | | | | | | | function in |
| | | | | | | | | humans |
| | | | | | | | | undercharacter |
| | | | | | | | | ized |
| | | | | | | | | (Accession |
| | | | | | | | | AC007563) |
| | TTCTCCTAGGAAGG | Intronic | TatD | 0% | 0% | 0% | 0% | (Accession |
| | AGGAAGAGG | (12q24.33) | TatDE | 0% | 0% | 0% | 0% | AC127071) |
| | AATCTTATGGCAGG | Intronic | TatD | 0% | 0% | 0% | 0% | (Accession |
| | AAGAAGAGG | (9q22.2- | TatDE | 0% | 0% | 0% | 0% | AL137847) |
| | | q31.1) | | | | | | |
| Tat | GAAGAAAACAAAGA | Intronic | TatE | 0% | 0% | 0% | 0% | (Accession |
| E | AGAAGGAGG | (18q12.2) | TatDE | 0% | 0% | 0% | 0% | AC118757) |
| | GAAGGAAGAGAAGA | Intronic | TatD | 0% | 0% | 0% | 0% | (Accession |
| | AGAAGGAGG | (19q13.33) | TatDE | 0% | 0% | 0% | 0% | AC010330) |
| | GAAGGAATCA- | Intronic | TatD | 0% | 0% | 0% | 0% | (Accession |
| | AGAAGAAGGAGG | (4q35.1) | TatDE | 0% | 0% | 0% | 0% | AC079080) |
| | GAAAGGAAGGAAG | Senescence | TatD | 0% | 0% | 0% | 0% | (Accession |
| | AAGAAGGGGG | gene | TatDE | 0% | 0% | 0% | 0% | AP000078.1) |
| | | region | | | | | | |
| | | (8p11.2) | | | | | | |
| | AAAGGAATCGAAGA | Intronic | TatD | 0% | 0% | 0% | 0% | (Accession |
| | AGAAGGGGG | (7q31.33) | | | | | | AC005521) |
| | | | TatDE | 0% | 0% | 0% | 0% | (Accession |
| | | | | | | | | AC005521) |

To establish proof-of-concept in HIV-infected leukocytes, TatD and TatE mosaic crRNAs as well as non-targeting and reference crRNA controls were cloned onto lentiviral expression vector pLentiCRISPR-RFP657 (Addgene #75162). To first assess CRISPR-Cas9 function, ACH2 T cells and U1 promonocytes bearing 1-2 copies of latent HIV-1 proviral DNA copies per cell were transfected with cloned lentiviral-CRISPR constructs. Subsequent stimulation of ACH2 T cells with TNFα induced viral rebound in control- and LTR-1/GagD-treated cultures but not in those receiving single- or dual TatD plus TatE treatments. U1 promonocytes were equally responsive to LTR-1/GagD and TatD/TatE treatments. HIV-1 proviral DNA excision was present in both cell types and confirmed by sequencing. These data in aggregate validate the TatDE CRISPR-Cas9 system for lentiviral delivery.

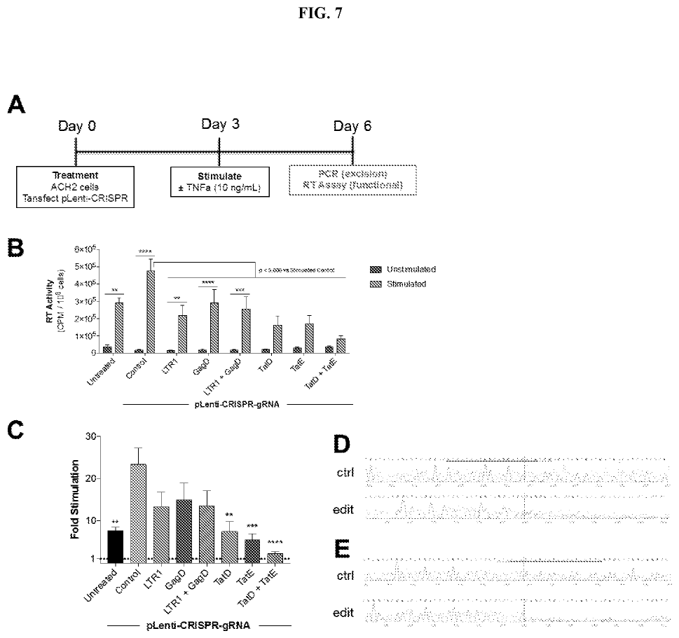

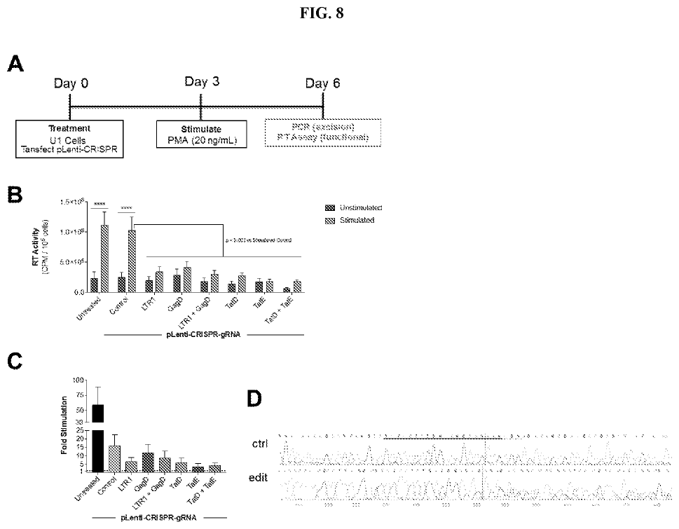

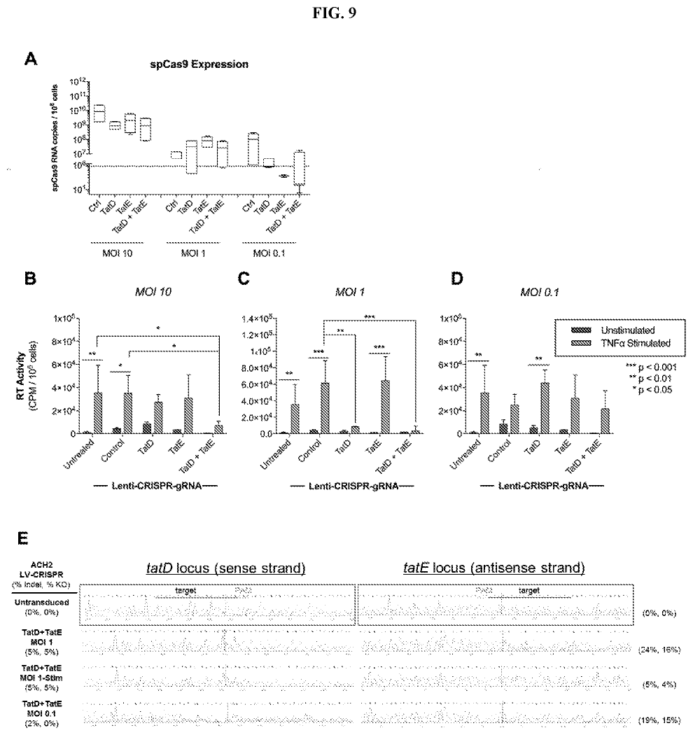

We next ascertained whether lentiviral transduction of TatDE CRISPR-Cas9 could halt HIV-1 induction from latently infected ACH2 T cells. Transgene expression was measured by reverse-transcriptase quantitative PCR (RT-qPCR) and determined to be above detection limits Transfection of LentiCRISPR plasmid constructs demonstrated superior reduction in preventing viral rebound in HIV-1 latently infected ACH2 T cells and U1 promonocytes (FIGS. 7-8). Dual TatD/TatE lentiviral treatments blocked TNFα-induced stimulation. Co-transduction of TatD/TatE at MOIs of 10 and 1 significantly reduced the release of HIV-1 into culture supernatants by 80% and 94%, respectively (FIG. 9).

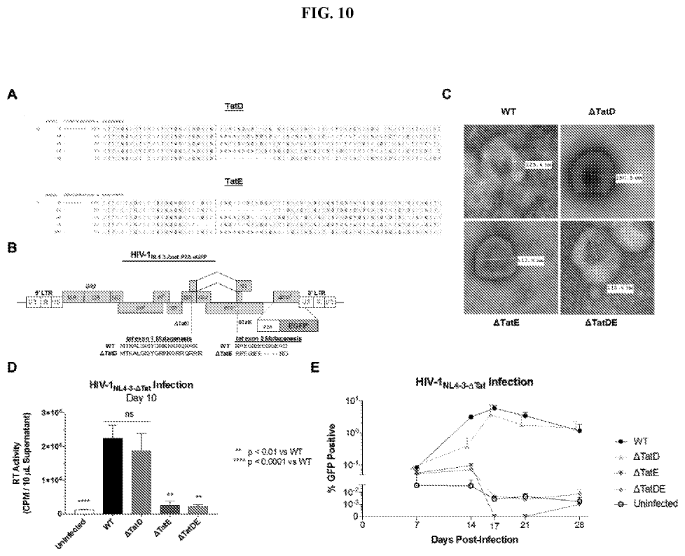

A concern of CRISPR editing of HIV-1 proviral DNA is the possibility of deriving escape mutants that a resistant to therapy. To evaluate such a possibility after disrupting 2 (TatD), 3 (TatE), or 5 (TatDE) viral exons, point mutants paralleling CRISPR edits were constructed and cultured for 28 days. Results demonstrate that exonic disruption, defined as interfering with 3 or more exons, halts viral replication (FIG. 10). We next screened dual mosaic crRNA constructs combining top single crRNA candidates to determine whether exonic disruption, length of excision, or sequence conservation most contributes to CRISPR antiviral efficacy (Table 5).

TABLE 5
Mosaic crRNA Coverage

| crRNA | % Sequence Conservation | Excised | Exons |
|---|---|---|---|
| Name | (Avg % Conservation) | Base Pairs | Disrupted |
| TatD | 59.04% | — | 2 |
| TatH^{§} | 51.12% | — | 1 |
| TatE | 15.26% | — | 3 |
| TatDH^{‡} | 59.44% (55.08%) | 29 | 3 |
| TatDE | 62.19% (37.15%) | 2442 | 5 |
| TatEH | 56.24% (33.19%) | 2451 | 4 |

^{§}4 base pairs of TatH crRNA overlap with rev. Given that CRISPR cleavage, as determined by ICE, occurs 5+ bp distal to PAM, TatH was considered to disrupt 1 exon (tat).
^{‡}Although TatDH is a composite of 2 highly conserved crRNAs, they overlap in target recognition and therefore multiplexing did not significantly increase breadth of coverage.

In an HIV-1 multistrain panel, TatDE which disrupts 5 exons (tat1-2/rev1-2/gp41) outperformed TatEH and TatDH constructs that represent the largest tested excision and most conserved single crRNAs, respectively (FIG. 5).

As a potential mechanism to explain TatE's high antiretroviral activity despite low sequence conservation, we investigated how disruption of different numbers of viral exons affects HIV-1 replicative fitness. Three HIV-1NL4-3-Δnef-eGFP non-frameshift point mutants were created to parallel CRISPR mutation profiles (FIG. 10A), in which two- (ΔTatD), three- (ΔTatE), or five (ΔTatDE) exons were altered (FIG. 10B). Resulting virions were imaged by transmission electron microscopy (TEM; FIG. 10C) and titered by RT-qPCR. The size of the HIV-1 tat mutants ranged from 110.6-150.5 nm in diameter, closely approximating that of wildtype HIV-1 measured at 125.4 nm. Likewise, the HIV-1 tat mutants' nearly spherical appearance and inner conical capsid matched the morphology of wildtype control virus. Next, CD4+T lymphocytes were infected at a MOI of 0.1. At 10 days following HIV-1 infection, significant differences in RT activity were detected in culture supernatants (FIG. 10D). HIV-1NL4-3-ΔtatD displayed nearly equivalent levels of viral replication as wildtype virus, while mutants bearing 3 or more disrupted exons were significantly lower in RT activity compared to unmutated control. As CD4+ T cell proliferation rates differed after 10 days due to HIV-1 induced cytopathicity, the percent of GFP-positive HIV-1 infected cells was measured by flow cytometry during a 28-day time course (FIG. 10E). Whereas HIV-1NL4-3-ΔtatD proliferation approximated that of control HIV-1, HIV-1NL4-3-ΔtatE and HIV-1NL4-3-ΔtatDE remained at or around baseline for four weeks. These data suggest that the locus targeted by TatE crRNA is critical for maximal CRISPR activity. In aggregate, we conclude that the high efficacy of TatDE CRISPR-Cas9 therapy against numerous HIV-1 strains results from disrupting five viral exons simultaneously.

Consensus sequence for whole HIV-1 genome (SEQ ID NO: 9):
>HIV1_ALL_2018_genome_DNA.fasta Consensus (threshold of >50%)
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NCNNNNNNNAAANNGAAAGNNNNNNNNNNNNNNNNNNNNNNNNNNNNNNNNNNNNN
NNNNNNNNNNNNNNNNNNNNNNNCCNNNNNNNAGNNNAGNNNANGNNNNNNNNNTN
CTCTNCNGACGCNANGGACTCGGCTTGCTGNNNNNNNNNANGNNNNGCNNNNCNNN
NCNGCAANNNNGAGGCGAGNGNNNNNGNNNNNNGCNGNCTGGTGNAGTACGNNNN
NNNNNCCNANNNNNNNNNNNNNNNNNNNNNNNNNNNNNNNNNNNNNNNNNNNNNNT
GACTANNNNNNNNNNNNNNNNNNNNNNNNNNNNGCGNNNGAGNNNNNNNNNNNNGC
TAGAAGGNNNNNNNNNNNNNNNAGANNNGAGNNNNNATGNNNGGTGCGAGAGCGTC
AGTANNNTTAAGNGGGGGANNNAAANNNTTAGATANATGGGAAAAAATTCGGTTAN
NNAGGCCANNNGGGGGAAAGNNNAAAAAATATANANTAAAACATNTAGTATGGGCA
AGCAGGGAGCTGGAAAGATTCGCACTTAACNNNNNNNNNNNNCCTGGCCTTTTAGA
AACANCAGAAGGCTGTAAACAAATAATGGAACAGCTACAACCAGCTCTTNNNCAGN
NNACAGGATCAGAAGAACTTAAATCATTATATAATACAGTAGCAACCCTCTATTGTG
TACATNNNCAAAGGATAGAGGTAAAAGACACCAAGGAAGCTTTAGANNNNNNNNNN
AAGATANNNNNNGAGGAAGAANNNNNNNNNNNNNNNNNNNNNCAANNNNNNNNNNN
NNNNNNNNNNNNNNNNNNNAACAAAAGTAAGNNNCAAAAGNCANNNNNNNNNNNNN
NNNNNNNNNNNNNNNNNNNNNNNNNNCAGCAAGCAGCAGCTNNNNNNNNNNNNNNN
NNNNNNNNNNNNNNNNNNNNNGNCACAGGAANCANCAGCAAGGTCNNNAGCNNNNN
NNNNNNNNNNNNNNNNCAAAATTACCCTATAGTGCAGAATCTNCAAGGGCAAATGG
TACATCAGNCCATATCACCTAGAACTTTAAATGCATGGGTAAAAGTAATANNNGAAG
AGAAGGCTTTCAGCCCAGAAGTAATACCCATGTTTTCAGCATTATCAGAAGGAGCC
ACCCCACAAGATNNNTTAAACACCATGCTAAACACAGTGNNNGGGGGACATCAANN
NGCAGCCATGCAAATGTTAAAAGANNNNACCATCAATGAGGAAGCTNNNGCAGAAT
GGGATAGATTACATNNNCCAGTACATGCAGGGCCTATTGCACCAGGCCAGATGNNN
AGAGAACCANNNAGGGGAAGTGACATAGCAGGAACTACTAGTACCCTTCAGGAACA
AATAGNATGGNNNATGACANNNNNNNNNNNNNNNNNNAGTNNNAATCCANNNCCTA
TCNNNNNNNNNCCAGTGGGAGAAATCNNNTATAAAAGATGGATAATCCTGGGATTA
AATAAAATAGTAAGAATGTATAGCCCTGTCAGCATTTTGGACATAAGACAAGGGCC
AAAGGAACCCTTTAGAGACTATGTAGACCGGTTCTTTAAAACTCTAAGAGCTGAGC
AAGCTACACAGGANGTANNNAAAAATTGGATGACAGAAACCTTGTTGGTCCAAAAT
GCGNNNAACCCAGATTGTAAGACCATTTTAAAAGCATTAGGACCAGGAGCTACATT
AGAAGAAATGATGACAGCATGTCAGGGAGTGGGAGGACCTGGCCATAAAGCANNN
AGAGTTTTGGCTNNNNNNNNNNNNNNNNNNGAGGCANNNNNNNNNATGAGCNNNNN
NNNNNNNNNNNNNNNNNNNNNNNNNNNNNNNCAAGNAANNNNNNNNNNNNNNNNNN
NNNNNNNNNNNNNNNNNNCNANCATANNNNNNATGATGNNNNNNNNNNNNNNNNNN
NNNNNNCAGAGANNNNNNNNNNNNNNNGGCAATTTTAAGNNNNNNGGCCAANNNAG
AAGANNNNNNNNNNNNANTNNNNTTNNNAAGTGTTTCAACTGTGGCAAAGAAGGGN
NNCACNNNATAGCCNNNNNNAGAAATTGCAGGGCCCCTAGGAAAAAGGGCTGTTGG
AAATGTGGAAAGGAAGGACACCAAATGAAAGACNNNNNNTGTNNNNNNNNNACTGA
GAGANNNCAGGCTNNNAATTTNTTTAGGGAAAATTTGGCCNNNTTCNNNCCANNNNN
NNNNCAANNNGGGNNNGAGNNNGCCAGGNNNNNNNNNNNNNNNNNNNNNNNNNNNN
NNNNNNNNGAATTTTCTTCAGAGNNNNNNNNNNNNNNNNNNNNNNNNNNNNNNNNNN
NNNNNNNNNNNNNNNNNNNNNNNNNNNNNNNNCAGACCNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNAGAGCCAACAGCNNNCCCNNNNNNNNNN
NNNNNACCAGCAGAGAGCTTCAGGTTNGGGGAGGAGACAACNNCNCCNNCNNNNNN
NNNNNNGAANNNNNNNNNNNNNNNNNNNNNNNNGCAGGAGCCGAAAGACAAGGANC
NNNNNNNNNNNNNNNNNNNNNNNNNNNNNNNNNNTCCNTTAGCTTCCCTCAAATCAC
TCTTTGGCAACGACCCCTNGTCACAATAAAAATANNNGGGGGNCAGCTAAAGGAAG
CTCTATTANNNGATACAGGAGCAGATGATACAGTATTANNNNNNGAAGAANNNNNN
NNNATAAATTTGCCAGGANNNAAATGGAAACCAAAAATGATAGGGGGAATTGGAGG
TTTTATCAAAGTAAGACAGTATNNNGATCAGATACCTATAGAAATTTGTGGANNNAA
AAAGGCTATANNNGGTACAGTATTAGTAGGACCTACACCTGTCAACNNNATAATTG
GAAGAAATATGTTGACTCAGATTGGTTGCACTTTAAATTTTCCAATTAGTCCTATTG
AAACTGTACCAGTAAAATTAAAGCCAGGAATGNNNGATGGCCCAAAGGTTAAACAA
NNNTGGCCATTGACAGAAGAANNNAAAATAAAAGCATTAACAGAAATTTGTACANN
NNNNGAANNNATGNNNGAAAAGGAAGGAAAAATTTCAAAANNNATTGGGCCTGAAA
ATCCATACAATACTCCAGTATTTGCCATAAAGAAAAAANNNNNNGACNNNNNNNNNN
NNAGTNNNNNNACTNNNAAANNNNNNTGGAGAAAATTAGTAGATTTCAGAGAACTN
AATAAAAGAACTCAAGACTTCTGGGAANNNGTTCAATTAGGAATACCACATCCNGC
AGGGTTANNNAAAAAGNNNAAANNNAAATCAGTAACAGTACTGGATGTGGGNGATG
CATATNNNTTTTCAGTTCCNTTAGATGAAGACTTCAGGAAGTATACTGCATTCACCA
TACCTAGTATAAACAATGAGACACCAGGGATTAGATATCAGTACAATGTGCTTCCAC
AGGGATGGAAAGGATCACCAGCAATATTCCAAAGTAGCATGNNNACAAAAATCTTA
GAGCCCTTTAGAANANNNCAAAATCCAGAAATAGTTATCTATCAATACATGGATGAT
TTGTATGTAGGATCTGACTTAGAAATANNNGGGCAGCATAGAACAAAAATAGAGGA
GCTAAGANNNNNNGAACATCTATTGANGTGGGGATTTACCACACCAGACNNNAAAA
AACATCAGAAAGAACCTCCATTTCTTTGGATGGGNTATGAACTCCATCCTGACAAAT
GGACAGTACAGCCTATANAGCTGCCAGAAAAAGACAGCNNNTGGACTGTCAATGAT
ATACAGNNNAAGNNNTTAGTGGGAAAATTANNNNNNNNNNNNNNNNNNAATNNNNN
NNNNNNNNNNNNNTGGGCAAGTCAGATTTATCCAGGGATTAAAGTAAAGCAATTAT
GTAAACTCCTTAGGGGAGCCAAAGCACTAACAGANATAGTACCACTAACTGAAGAA
GCAGAATTAGAATTGNNNGCAGAAAACAGGNNNGAAATTCTAAAAGAACCAGTACA
TGGAGTATATTATGACCCATCAAAAGACTTAATAGCAGAANNNATANNNNNNCAGAA
ACAGGGGCAAGACCAATGGNNNNNNNNNNNNNNNNNNNNNNNNNNNACATATCAAA
TTTATCAAGAGCCATTTNNNAAAAATCTGAAAACAGGANNNAAATATGCANNNAGAA
TGAGGNCTNNNGCCCACACTAATGATGTAAAACAATTAACAGAGGCAGTGCAAAAA
ATAGCCACAGAAAGCATAGTAATATGGGGAAAGNNNACTCCTAAATTTAGACTACC
CATACAAAAAGAAACATGGGAANNNNNNACATGGTGGACAGAGTATTGGCAAGCCA
CCTGGATTCCTGAGTGGGAGTTTGTCAATACCCCTCCCCTAGTAAAATTATGGTACC
AGTTAGAANNNAAAGAANNNNNNNNNNNNNNNCCCATAGTAGGAGCAGAANNNACT
TTCTATGTAGATGGGGCAGCTAATNNNAGGGANACTAAANTAGGAAAAGCAGGATA
TGTTACTGACAGAGGAAGACAAAAAGTTGTTTCCCTAACTGANACAACAAATCAGA
AGACTGAATTACAAGCAATTCATCTAGCTTTGCAGGATTCAGGATCAGAAGTAAACA
TAGTAACAGACTCACAATATGCATTAGGAATCATTCAAGCACAACCAGATAAGAGT
GAATCAGAGTTAGTCAATCAAATAATAGAGCAGTTAATAAAANNNAAGGAAAAGGT
CTACCTGTCATGGGTACCAGCACACAAAGGAATTNNNGGAGGAAATGAACAAGTAG
ATAAATTAGTCAGTACTNNNGGAATCAGGAAAGTACTATTTTTAGATGGAATAGATN
NNAAGGCTCAAGAANNNGANCATGAAAAATATCACAGCAATTGGAGAGCAATGGCT
AGTGATTTTAATCTGCCACCTATAGTANNNGCAAAAGAAATAGTAGCCNNNAGCTGT
GATAAANNNTGTNNNCAGCTAAAAGGGGAANNNGCCATGCATGGACAAGTAGACTG
TAGTCCAGGAATATGGCAATTAGATNNNTGTACACATTTAGAAGGAAAAATTATCCT
GGTAGCAGTCCATGTAGCCAGTGGNTATATAGAAGCAGAAGTTATCCCAGCAGAAA
CAGGACAGGAAACAGCATACTTTATNTTAAAATTAGCAGGAAGATGGCCAGTAAAA
GTAATACATACAGACNNNAATGGCAGCAATTTCACCAGTGCTGCAGTTAAGGCAGC
CNNNTGTTGGTGGGCAGGTATCAAACAGGAATTTGGNATTCCCTACAATCCCCAAA
GTCAAGGAGTAGTAGAATCTATGAATAAAGAATTANNNAAGAAAATTATAGGNCAG
GTAAGAGATCAAGCTGAACACCTTAAGACAGCAGTACAAATGGCAGTATTCATTCA
CAATTTTAAANNNAGAAAANNNGGGGGGATTGGGGGGTACAGTGCAGGGNNNNNNN
NNNNNNNNNNNNNNNNNNNNNNNNNNNNNNNNNNNNNNNNNNNNGAAAGAATAATA
GACATAATAGCAACAGACATACAAACTAAAGAATTACAAAAANNNCAANNNATTACA
NNNAAAATTNNNCAAAATTTTCGGGTTTATTACAGGGACAGCAGAGACCCAATTTGG
AAAGGACCAGCAAAACTACTCTGGAAAGGTGAAGGGGCAGTAGTAATANNNNNNCA
ANNNNNNNNNNNNGATAATNNNNNNAGTGACATAAAAGTAGTACCAAGAAGAAAAG
CAAAGATCATTAGGNNNGATTATGGAAAACAGATGGCANNNGGTGATGATTGTGTG
NNNGCANNNGGTAGACAGGATGAGGATTNNAGANNNACATGGAANAGTTTAGTANN
NAAACACCATATGTATATTTCAAAGAAAGCTAANGGATGGTTTTATAGACATCACTA
TGAAAGCAGNNNNCATCCANNNAAAGTAAGTTCAGAAGTACACATCCCACTAGGGG
ANNNNNNNNNNNNNNNNGCTANATTAGTAATAANAACATATTGGNNNGGTCTGCATA
CAGGAGAAAGAGANTGGCATTTGNNNGGTCATGGAGTCTCCATAGAANNNTGGAGG
NAGAGAAGATATNNNAGCACACAAGTAGACCCTGACCTAGCAGACCAACTAATTCA
TCTGCATTATNNNTTTGATTGTTTTTCAGANTCTGCCATAAGGNNNAAAGCCATATT
AGGACANNNNATAGTTAGNCCTAGGTGTGAATATCAAGCAGGANNNCATAACNNNA
AGGTAGGATCTCTACAATACTTGGCACTNACAGCATTAATANNNNNNNNNNNNNNNA
CACCAAAAAAGANAAAGCCACCTTTGCCTAGTGTTANGAAATTAACAGAGGATAGA
NNNNNNNNNNNNNNNTGGAACAAGCCCCAGNNNAAGACCANGGGCCACAGAGGGA
NCCATACAATGAATGGACACTAGNAGCTTTTAGAGGAGCTTAAGAATGAAGCTGTT
AGACATTTTCCTAGGCCATGGNNNCTCCATGGCTTAGGACAATATATCTATGAAACT
TATGGGGATACTTGGGCAGGAGTTGAAGCTNNNATAATANNNAGAATTCTGCAACA
ACTACTGTTTATTCATNNNTTCNNNAGAATTGGGTGTCAANNNCATAGCAGAATAGG
CATTATTCGANNNNNNNNNNNNCAGAGAAGANNNNNNNNNNNNNNNGCAAGAAATG
GAGCCAGTNNNNNNAGATCCTAANNCCTAGAGCCCTGGNNNNNNNNNNNNAANCAT
NNNCCAGGAAGTCAGCCTAAAACTNNNGCTTGTANCAANNNNNNNNNNTGCTATTGT
AAAAAGTGTTGCTNTNNNCATTGCCAANNNNNNNNNNNNNNNGTTTGCTTTNTGNNN
AAAAAAGGCTTANNNNNNNNNGGCATCTCCTATGGCAGGAAGAAGCGGAGACAGCG
ACGAAGANNNNNNNNNNCTCCTCAAAGCAGTNAGGATCATCAAAATCCTNTATCAAA
GCANNNNNNNNNNNNNNNNNGTAAGTANTANNNNNNNNNNNNNNNNNNNNNNNNNN
NNNNNNNNNNNNNTGTAATGNAANCTTTANAAATANTAGCANNNATAGTAGCATTAG
TAGTAGCANTANNNATANNNATAGCAATAGTTGTGTGGACCATAGTATNTATAGAAN
NNNNNNNNNNNTATAGGAAAATANTAAGACAAAGAAAAATAGACAGGTTAATTGAN
NNNNNNNNNNNNAGAATAAGANNNGAAAGAGCAGAAGACAGTGGCNNNNNNNNNAN
NATGNNNAGAGTGANGGGGATACAGNNNNNNNNNNNNNNNNNNNNNNNNNNNNNNN
NNANGAATTGTCANCACTTGTGGANATGGGGCATCTTGNTCCTTGGGATGTTNATG
ATNTGTNNNNNNNNNNNNNNNNNNNNNNNNNNNNNNAGTGCTGCAGAAAACTTGTG
GGTCACAGTCTATTATGGGGTACCTGTGTGGAAAGAAGCANNNAANACCACTNNNN
NNCTATTTTGTGCATCAGATGCTAAAGCATATGANACAGAAGTACATAATGTCTGGG
CNACACATGCCTGTGTACCCACAGACCCCAACCCACAAGAAATANNNTTGGAANNN
NNNAATGTAACAGAAAATTTTAACATGTGGNNNAAAAATAACATGGTAGANCAGAT
GCATGAGGATATAATCAGTTTATGGGATCAAAGCCTAAAGCCATGTGTAAAGTTAA
CCCCACTCTGTGTTACTTTAAATTGTACTAATGNNAANANNANTANTANTANTANNA
NNANNANNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNGNAGAAATAAAAAACTGCTCTTTCAATATNACCNNNACAGAAATAAGAGA
TAAGAAGCAGAAANNNNNNNNNGNATATGCACTTTTTTATAAACTTGATATAGTACC
AATNGATNATAATNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNANTANTANNTATAGNTTAATAAATTGTNNNAATACCNNNTCAGCCNNNATTN
NNNNNACACAGGCCTGTCCAAAGGTATCCTTTNNNGANNNNNNNNNNNNNCCAATTC
CCATACATTATTGTGCNCCAGCTGGTTTTGCGATTCTAAAGTGTNNNAATNNNGATA
AGAANTTCAATNNNNNNGGANNNACAGGACCATGTNNNAAAAATGTCAGCACAGTA
CAATGTACACATNNNGGAATTAAGCCAGTAGTATCANNNACTCAANNNCTGCTGTTA
NNNAATGGCAGTCTAGCAGAANNNNNNNNNNNNNNNNNNNNNNNNNNNNNNNNNGA
ANNNGAGATAATAATTAGATCTGAAAATNNNNNNNTCACAAACAATNNNNNNNNNGC
NAAAACCATAATAGTACANCTTAATGAANNNNNNTCTNNNGTAGAAATTAATTGTAC
AAGACCCAACAACAATACAAGAAAAAGTATACNTATANNNNNNGGACCAGGACAAN
NNNNNNNNGCATTCTATGCANNNNNNNNNNNNACAGGAGANATAATAGGAGATATA
AGACAAGCACATTGTAACATTAGTAGAACANAANNNNNNTGGAATAAAACTTTANAA
CAGGTAGNTAAAAAATTAAGAGAANNNNNNNNNNNNNNNNNNNNNNNNNNNNNNCA
NTTNNNTAATAAAACANNNNNNNNNNNNNNNNNNNNNNNNNNNNNNATAANCTTTNN
NNNNAANCNATCCTCAGGAGGGGACCTAGAAATTACAACNCATAGTNNNTTTAATTG
TGGAGGAGAATTTTTCTATTGTAATACATCANAACTGTTTAATAGTACTTGGANTNN
NNNTANTNNNANNNNNNNNNNNNNNNNNNNNNNNNNNNNNNNNNNNNNNNNNNNNN
NNNNNNNNNNNNNNNNNNNNNNNNNNNNNAATGNNACTATCACACTCCCATGCAGA
ATAAAACAAATTATAAACATGTGGCAGGAAGTAGGACAAGCANNNATGTATGCCCC
TCCCATCNNAGGAAAAATTANATGTNNATCAAATATTACAGGACTACTATTAACAAG
AGATGGTGGNAATAANAANNNNNNNNNNNNNNNNNNNNNNNNNNNNNNNNNNNNNN
NNNNNNNNNNNNNNAATANNANNGAGACCTTCAGACCTGGAGGAGGAGATATGAGG
GACAATTGGAGANNNNNNAGTGAATTATATAAATATAAAGTAGTAAAAATTGAACCA
TTANNNGGAGTAGCACCCNNNNNNACCAAGGCAAAGAGAAGAGTGNNNNNNNNNNN
NNNNNNNGTGGAGAGAGAAAAAAGAGCANNNNNNGTNGGAATAGGANNNGCTNTGT
TCCTTGGGTTCTTGGGAGCAGCAGGAAGCACTATGGGCGCAGCGTCAATAACGCTG
ACGGTACAGGCCAGACAATTATTGNNNTCTGGTATAGTGCAANNNCAGCAANNNAG
CAATTTGCTGAGGGCTATAGAGGCGCAACAGCATCTGTTGCAACTCACAGTCTGGG
GCATTAAACAGCTCCAGNNNGCAAGAGTCCTGGCTGTGGAAAGATACCTAAAGNNN
GATCAACAGCTCCTAGGGATTTGGGGCTGCNNNNNNTCTGGAAAACTCATCTGCAC
CNNNACTACTGTGCCTTGGAACNNNNCTAGTTGGNNNNNNNNNNNNNNNNNNNNNN
NNNNNNNNNNNAGTAATNNNNNNNNNNNNAAATCTCANGATGANATTTGGGATNNNN
NNNNNAACATGACCTGGATGCAGTGGGANNNNAGAGAAATTAACAATTACACANAC
NNAATATACANNTTACTTGAAGAATCGCAAAACCAGCAGGAANNNAAGAATGAACA
AGANTTATTGGCATTGGACAANTGGGCAAGTCTGTGGAATTGGTTTGACATAACAA
ANTGGCTGTGGTATATAAAAATATTCATAATGATAGTAGGAGGCTTAATAGGTTTAA
GAATANNNGTTTTTGCTGTGCTTTCTATAGTAAATAGAGTTAGGCAGGGATACTCAC
CTTTGTCGTTNCAGACCNNNCNTATCCCANNCCCGAGGGGANNNCCCNNNNNNGAC
AGGCCCGAAGGAATCGAAGAAGAAGGTNNNGGAGAGCAAGACAGAGACAGATCCA
TNCGATTAGTGANCGGATTCTTAGCACTTGTCTGGGACGACCTGCGGAGCCTGTGC
CTCTTCAGCTACCACCGCTTGAGAGACTTANTCTTGATTGCAGCGAGGACTGTGGA
ACTTCTGGGACGCAGNNNNNNNNNNNNNNNNNNNNNNNNNNNNNNNGGGTGGGAAG
NCCTCNNNNNNNNNAAATATCTGTGGAATCTNCTGCAGTATTGGGGTCAGGAACTA
NNNAAGAATAGTGCTATTAGTTTGCTTGATACCANAGCAATAGCAGTAGCTGAGGG
GACAGATAGGATTATAGAAGTAGTACAAAGANTTTGTAGAGCTATTCTCCACATACC
TAGANNNNNNNNNAGAATAAGACAGGGCTTNGAAAGNNNNNNNNNNNNNNNNNNNN
NNNNNNNNNNNNNNNNNNNNGCTTTGCTATAANNNNNNNNNNNNNNNNNNNNNNNNN
NNNNNNNNNNNNNNNNNNNNATGGGNGGCAAGTGGTCAAAAAGTAGNNNNNNNNNN
NNNNNNATAGTNGGATGGCCTNNNNNNNNNNNNNNNNNNNNNNNNNNNGCNNNNGT
AAGGGAAAGAATGAGACGANNNNNNNNNNNNNNNNNNNNNNNNNNNNNNNNNNNNN
NNNNNNNNNNNNNNNNNNNNNNNNCTGANCCAGCAGCAGANGGAGTAGGAGCAGTA
TCTCAAGACTTAGANAAACATGGAGCAATCACAAGTAGCAATACANNNGCNNNNAA
TAATGCTGATTGTGCCTGGCTGGAAGCACAANNNNNNNNNNNNGAGGANGAGGANG
TAGGCTTTCCAGTCAGANNNNNNCCTCAGGTACCTTTAAGANNNCCAATGNNNACTT
ATAAGGGAGCTTTNNNNGATCTNAGCNNNNNCTTTTTANNNAAAGAAAAGNNNGGG
GGACTGGAAGGGNTAATTTACTCCAAGNNNAAAAGANNNNNNCAAGANATCCTTGA
TCTGTGGGTCTATCACACACAAGGCTACTTCNNNCCTGATTGGCANAACTACACACC
AGGGCCAGGGATCAGANNNTANCCACTGACCTTTGGATGGTGCTTCAAGCTAGTAC
CAGTTGANNNNCCAGANGANGTANNNGAAGAGNNNGCCAATGAAGGAGAGAACAAC
TGCTTGNNNNNNNNNNNNNNNNNNNNNNNNTTACACCCTATGAGCCAGNNNCATGG
AATGGANGANGNANNNNNNGANAGAGAAGTGTTAATGTGGAAGTTTGACAGCNNCC
TAGCANNNNNNANACACATNGCCCGAGAGNNNCTGCATCCGGAGTNCTACNNNNNN
NNNNNNNNNNNNNNNNNNNNNNNNNNNNNNNNNNNNNNNAAAGACTGCTGANNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNAGGGACTTTCCNNNNNNNNNNNNNNNNNNNNNNNNCNGGGGACTTTCCNNN
NNNNNNNNANNNNNNNNNNNNNNNNNNNNNNGGNNNNNNNNNNNNNNNNNNNGGNN
GTGGNNNNNNGNNGGCGNNNNNNNNNNGNNNNNNNNNNNNNNNNNNNNNNNGGGG
NAGTGGNCNANCCCNNTCAGNANNNNNTGCTGNNNCATATAAGCAGCNGCTTTTNN
GCNNTGTACTNGGGTCTCNNNNNNTCNNNTNGNNNNNNNGACCAGNTNNGAGCCNG
GGNANCTCNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
NNNNNNNNNNNNNNNNNNNNNN
Consensus sequence for HIV-1 Tat exon 1 (SEQ ID NO: 10):
ATGGAGCCAGTNNNNNNAGATCCTAANNCCTAGAGCCCTGGNNNNNNNNNNNNAAN
CATNNNCCAGGAAGTCAGCCTAAAACTNNNGCTTGTANCAANNNNNNNNNNTGCTAT
TGTAAAAAGTGTTGCTNTNNNCATTGCCAANNNNNNNNNNNNNNNGTTTGCTTTNTG
NNNAAAAAAGGCTTANNNNNNNNNGGCATCTCCTATGGCAGGAAGAAGCGGAGACA
GCGACGAAGANNNNNNNNNNCTCCTCAAAGCAGTNAGGATCATCAAAATCCTNTAT
CAAAGCA
Consensus sequence for HIV-1 Tat exon 2 (SEQ ID NO: 11):
ACCNNNCNTATCCCANNCCCGAGGGGANNNCCCNNNNNNGACAGGCCCGAAGGAAT
CGAAGAAGAAGGTNNNGGAGAGCAAGACAGAGACAGATCCATNCGATTAG

What is claimed is:

1. A nucleic acid sequence comprising a crRNA sequence that is complementary to a plurality of nucleic acids of a consensus sequence of an HIV-1 gene selected from the group consisting of: tat, rev, env-gp41, gag-p1, gag-p6, vif, vpr, vpu, and nef.

2. The nucleic acid sequence of claim 1, wherein the nucleic acid sequence comprises two crRNA sequences, each sequence complementary to a plurality of nucleic acids of a consensus sequence of an HIV-1 gene selected from the group consisting of: tat, rev, env-gp41, gag-p1, gag-p6, vif, vpr, vpu, and nef; wherein the crRNA sequences are not complementary to the same sequences.

3. The nucleic acid sequence of claim 1, wherein the crRNA sequence is adjacent to a PAM sequence.

4. The nucleic acid of claim 1, wherein the crRNA sequence is complementary to a plurality of nucleic acids of an overlapping sequence.

5. The nucleic acid of claim 1, wherein the overlapping sequence is part of a nucleic acid sequence of at least two HIV-1 genes selected from the group consisting of: tat, rev, env-gp41, gag-p1, gag-p6, vif, vpr, vpu, and nef.

6. The nucleic acid of claim 1, wherein the overlapping sequence is part of a nucleic acid sequence of at least three HIV-1 genes selected from the group consisting of: tat, rev, env-gp41, gag-p1, gag-p6, vif, vpr, vpu, and nef.

7. The method of claim 5 or 6, wherein the overlapping exon is part of a nucleic acid sequence selected from the group consisting of tat (exon 1, nucleic acids 5831-6045; exon 2, nucleic acids 8379-8469), rev (exon 1, nucleic acids 5970-6045; or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417).

8. The nucleic acid of claim 4, wherein the overlapping sequence is nucleic acids 7758-8795 of HIV-1 gene gp41-env, exon 2 (nucleic acids 8379-8469) of HIV-1 gene tat, and exon 2 (nucleic acids 8379-8653) of HIV-1 gene rev.

9. The nucleic acid of claim 1, wherein the overlapping exon is exon 1 (nucleic acids 5831-6045) of HIV-1 gene tat, and exon 1 (nucleic acids 5970-6045) of HIV-1 gene rev.

10. The nucleic acid sequence of claim 1, wherein the crRNA has a sequence at least 80%, 85%, 90%, or 95% identical to SEQ ID NO: 1.

11. The nucleic acid sequence of claim 1, wherein the crRNA has a sequence at least 80%, 85%, 90%, or 95% identical to SEQ ID NO: 2.

12. The nucleic acid sequence of claim 1, wherein the crRNA has a sequence at least 80%, 85%, 90%, or 95% identical to SEQ ID NO: 3.

13. The nucleic acid sequence of claim 1, wherein the crRNA has a sequence at least 80%, 85%, 90%, or 95% identical to SEQ ID NO: 4.

14. The nucleic acid sequence of claim 1, wherein the crRNA has a sequence at least 80%, 85%, 90%, or 95% identical to SEQ ID NO: 5.

15. The nucleic acid sequence of claim 1, wherein the crRNA has a sequence at least 80%, 85%, 90%, or 95% identical to SEQ ID NO: 6.

16. The nucleic acid sequence of claim 1, wherein the crRNA has a sequence at least 80%, 85%, 90%, or 95% identical to SEQ ID NO: 7.

17. The nucleic acid sequence of claim 1, wherein the crRNA has a sequence at least 80%, 85%, 90%, or 95% identical to SEQ ID NO: 8.

18. The nucleic acid sequence of claim 1, wherein the crRNA has a sequence according to SEQ ID NO: 1.

19. The nucleic acid sequence of claim 1, wherein the crRNA has a sequence according to SEQ ID NO: 2.

20. The nucleic acid sequence of claim 1, wherein the crRNA has a sequence according to SEQ ID NO: 3.

21. The nucleic acid sequence of claim 1, wherein the crRNA has a sequence according to SEQ ID NO: 4.

22. The nucleic acid sequence of claim 1, wherein the crRNA has a sequence according to SEQ ID NO: 5.

23. The nucleic acid sequence of claim 1, wherein the crRNA has a sequence according to SEQ ID NO: 6.

24. The nucleic acid sequence of claim 1, wherein the crRNA has a sequence according to SEQ ID NO: 7.

25. The nucleic acid sequence of claim 1, wherein the crRNA has a sequence according to SEQ ID NO: 8.

26. The nucleic acid sequence of one of claims 1-25, wherein the nucleic acid sequence further comprises a tracrRNA sequence.

27. The nucleic acid sequence of any one of claims 1-26, wherein the nucleic acid sequence further comprises a sequence that encodes a Cas protein.

28. The nucleic acid of claim 27, wherein the Cas protein is a Cas9, CasPhi (Cas Φ), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas12a (Cpf1), Cas12b, Cas12e, Cas13a, Cas13, Cas13c, or Cas13d.

29. The nucleic acid of claim 27, wherein the Cas protein is a Cas9 protein.

30. The nucleic acid of any one of claims 1-29, wherein the nucleic acid sequence is a DNA sequence.

31. The nucleic acid of any one of claims 1-29, wherein the nucleic acid sequence is a RNA sequence.

32. The nucleic acid of any one of claims 1-29, wherein the nucleic acid sequence further encodes a viral vector.

33. The nucleic acid of claim 32, wherein the viral vector is an adenovirus, an adeno-associated virus (AAV), a retrovirus, or a herpesvirus.

34. The nucleic acid of claim 32, wherein the viral vector is AAV-1, AAV-2, AAV-3, AAV-4, AAV-5, AAV-6, AAV-7, AAV-8, AAV-9, AAV-10, AAV-11, AAV-12, AAV-13 or AAV rh.74.

35. The nucleic acid of claim 32, wherein the viral vector is a lentivirus.

36. The nucleic acid of claim 32, wherein the viral vector is a HIV, FLV, MLV, mMLV, VSV-G enveloped lentivirus, or HIV-enveloped lentivirus.

37. The nucleic acid of claim 32, wherein the viral vector is herpes simplex I virus (HSV).

38. The nucleic acid of claim 32, wherein the nucleic acid further comprises a eukaryotic promoter operably connected to the crRNA sequence.

39. The nucleic acid of claim 32, wherein the nucleic acid comprises tracrRNA sequence and a eukaryotic promoter operably connected to the tracrRNA sequence.

40. The nucleic acid of claim 39, wherein the tracrRNA is operably connected to the crRNA to form a sgRNA.

41. The nucleic acid of claim 31, wherein the nucleic acid comprises a sequence encoding a Cas protein and a eukaryotic promoter operably connected to the Cas protein sequence.

42. The nucleic acid of claim 41, wherein the Cas protein is a Cas9 protein.

43. The nucleic acid of any one of claims 38-42, wherein the eukaryotic promoter is a cytomegalovirus (CMV) promoter or EF-1 alpha.

44. A pharmaceutical composition comprising (a) a nucleic acid according to any one of claims 1-34, and (b) a pharmaceutically acceptable excipient.

45. The pharmaceutical composition of claim 44, comprising (a) a nucleic acid comprising TatD and (b) a nucleic acid comprising TatH (TatD/H).

46. The pharmaceutical composition of claim 44, comprising (a) a nucleic acid comprising TatD and (b) a nucleic acid comprising TatE (TatD/E).

47. The pharmaceutical composition of claim 44, comprising (a) a nucleic acid comprising TatE and (b) a nucleic acid comprising TatH (TatE/H).

48. The pharmaceutical composition of claim 44, comprising (a) a nucleic acid comprising TatD and (b) a nucleic acid comprising TatA₂ (TatA₂/D).

49. The pharmaceutical composition of claim 44, comprising a nucleic acid comprising TatD/tracrRNA and (b) a nucleic acid comprising TatH/tracrRNA.

50. The pharmaceutical composition of claim 44, comprising (a) a nucleic acid comprising TatD/tracrRNA and (b) a nucleic acid comprising TatE/tracrRNA.

51. The pharmaceutical composition of claim 44, comprising (a) a nucleic acid comprising TatE/tracrRNA and (b) a nucleic acid comprising TatH/tracrRNA.

52. The pharmaceutical composition of claim 44, comprising (a) a nucleic acid comprising TatD/tracrRNA and (b) a nucleic acid comprising TatA₂/tracrRNA.

53. A method of disrupting the transcription of an exon of an HIV-1 sequence in an individual in need thereof, comprising administering to the individual a nucleic acid according to any one of claims 1-43.

54. A method of excising all or a portion of an HIV-1 sequence in an individual in need thereof, comprising administering to the individual a nucleic acid according to any one of claims 1-43.

55. A method of treating an HIV-1 infection in an individual in need thereof, comprising administering to the individual a nucleic acid according to any one of claims 1-43.

56. A method of preventing an HIV-1 infection in an individual in need thereof, comprising prophylactically administering to the individual a nucleic acid according to any one of claims 1-43.

57. A method of preventing transmission of an HIV-1 virus from a first individual to a second individual, comprising administering to the first individual a nucleic acid according to any one of claims 1-43.

58. The method of claim 57, wherein the first individual is a pregnant woman and the second individual is a child.

59. The method of any one of claims 53-58, comprising administering to the individual (a) a nucleic acid comprising TatD and (b) a nucleic acid comprising TatH (TatD/H).

60. The method of any one of claims 53-58, comprising administering to the individual: (a) a nucleic acid comprising TatD and (b) a nucleic acid comprising TatE (TatD/E).

61. The method of any one of claims 53-58, comprising administering to the individual: (a) a nucleic acid comprising TatE and (b) a nucleic acid comprising TatH (TatE/H).

62. The method of any one of claims 53-58, comprising administering to the individual: (a) a nucleic acid comprising TatD and (b) a nucleic acid comprising TatA₂ (TatA₂/D).

63. The method of any one of claims 53-58, comprising administering to the individual (a) a nucleic acid comprising TatD/tracrRNA and (b) a nucleic acid comprising TatH/tracrRNA.

64. The method of any one of claims 53-58, comprising administering to the individual (a) a nucleic acid comprising TatD/tracrRNA and (b) a nucleic acid comprising TatE/tracrRNA.

65. The method of any one of claims 53-58, comprising administering to the individual (a) a nucleic acid comprising TatE/tracrRNA and (b) a nucleic acid comprising TatH/tracrRNA.

66. The method of any one of claims 53-58, comprising administering to the individual (a) a nucleic acid comprising TatD/tracrRNA and (b) a nucleic acid comprising TatA₂/tracrRNA.

67. The method of any one of claims 53-66, wherein a first viral vector comprises the crRNA sequence and the tracrRNA sequence.

68. The method of claim 67, wherein the first viral vector comprises a second crRNA sequence, provided that the each crRNA sequence is complementary to a different target sequence.

69. The method of claim 67, wherein the crRNA sequence and the tracrRNA sequence are a sgRNA.

70. The method of any one of claims 53-69, wherein a second viral vector comprises the nucleic acid sequences encoding the Cas protein.

71. The method of claim 70, wherein the Cas protein is a Cas9, CasPhi (Cas Φ), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas12a (Cpf1), Cas12b, Cas12e, Cas13a, Cas13, Cas13c, or Cas13d.

72. The method of claim 70, wherein the Cas protein is a Cas9 protein.

73. The method of any one of claims 53-72, wherein the nucleic acid sequence further encodes a viral vector.

74. The method of claim 73, wherein the viral vector is an adenovirus, an adeno-associated virus (AAV), a retrovirus, or a herpesvirus.

75. The method of claim 73, wherein the viral vector is AAV-1, AAV-2, AAV-3, AAV-4, AAV-5, AAV-6, AAV-7, AAV-8, AAV-9, AAV-10, AAV-11, AAV-12, AAV-13 or AAV rh.74.

76. The method of claim 73, wherein the viral vector is a lentivirus.

77. The method of claim 73, wherein the viral vector is a HIV, FLV, MLV, mMLV, VSV-G enveloped lentivirus, or HIV-enveloped lentivirus.

78. The method of claim 73, wherein the viral vector is herpes simplex I virus (HSV).

79. The method of claim 73, wherein the nucleic acid further comprises a eukaryotic promoter operably connected to the crRNA sequence.

80. The method of claim 73, wherein the nucleic acid further comprises a eukaryotic promoter operably connected to the sgRNA sequence.

81. The method of claim 73, wherein the nucleic acid further comprises a eukaryotic promoter operably connected to the nucleic acid sequence encoding the Cas protein.

82. The method of any one of claims 79-81, wherein the eukaryotic promoter is a cytomegalovirus (CMV) promoter or EF-1 alpha.
